(19) United States
(12) Patent Application Publication
(10) Pub. No.: US 2020/0270678 A1
(43) Pub. Date: Aug. 27, 2020

(54) PERSONALIZED THERAPEUTIC APPROACHES TO PROSTATE CANCER

(71) Applicant: THE ROYAL INSTITUTION FOR THE ADVANCEMENT OF LEARNING/MCGILL UNIVERSITY, Montreal (CA)

(72) Inventors: Jacques LAPOINTE, Lasalle (CA); Yogesh M. BRAMHECHA, Halifax (CA)

(21) Appl. No.: 16/592,867

(22) Filed: Dec. 18, 2019

Related U.S. Application Data
(60) Provisional application No. 62/741,654, filed on Oct. 5, 2018.

(51) Int. Cl. C12Q 1/6841; C12Q 1/6886

(52) U.S. Cl. CPC C12Q 1/6841; C12Q 2600/158; G01N 2800/52; C12Q 1/6886

(57) ABSTRACT

Methods and kits are provided for assessing the cancer risk of a subject having prostate cancer by detecting DNA copy number alterations. In particular, 16p13.3 gain, and further in addition PTEN deletion are assessed, and detected using probes such as FISH probes to provide a customized treatment options to the screened individual.

### CROSS-REFERENCE TO PUBLICLY FOUNDED RESEARCH AND PREVIOUSLY-FILED APPLICATIONS

The present application claims priority from U.S. provisional application 62/741,654 filed on Oct. 5, 2018 and herewith incorporated in its entirety.

This invention was made with government support under W81XWH-11-1-0638 awarded by the Department of Defense. The government has certain rights in the invention.

### FIELD OF THE INVENTION

The present disclosure relates to the use of genetic biomarkers for prognosing prostate cancer and personalizing therapeutic approaches.

### BACKGROUND

Prostate cancer remains a major clinical burden, being the most prevalent cancer and one of the leading causes of cancer-specific deaths in North American men. It is a clinically heterogeneous disease wherein the majority of cancers display a favorable outcome, while a subset affecting a considerable number of patients progress to metastatic and lethal stage. Radical prostatectomy (RP) or radiation therapy is considered the standard primary treatment option for localized prostate cancer and more recently, active surveillance has emerged as a viable alternative for patients presenting favorable clinicopathologic features.

One of the key challenges in the clinical management of prostate cancer is to accurately distinguish indolent from aggressive tumors in order to avoid overtreatment of clinically insignificant cancers and undertreatment of tumors with metastatic potential. Serum prostate-specific antigen (PSA) levels, biopsy Gleason grade (GS) and clinical tumor stage (cT-stage) are used to risk stratify patients, but are not sufficient to accurately predict individual clinical outcome. Assessing the Gleason grade based on prostate biopsies is challenging and frequently leads to an underestimation of the actual grade of the entire tumor burden. In particular, precisely assessing GS on biopsies is limited by the fact that partial sampling may result in an underestimation of the final score of cancer in the RP specimen. The majority of patients undergoing RP present low-intermediate risk clinical features, and accurate prognosis within this subgroup of patients still remains a clinical challenge. Moreover, although most patients respond well to RP, a significant proportion will experience a disease recurrence, as assessed by a rise in serum PSA that might eventually progress to the metastatic stage. Early identification and more accurate risk stratification may, therefore, allow patients with aggressive tumors to receive appropriate treatment without delay while sparing patients with clinically favorable tumors from treatment side effects.

To address the shortcomings of the clinicopathologic predictors and to better capture the clinical heterogeneity of prostate cancer, improved biomarkers are needed.

### SUMMARY

The present application concerns methods for prognosing and treating prostate cancer using 16p13.3 and/or 10q23.3 (PTEN) biomarkers.

In a first aspect, the present disclosure provides a method of prognosing a subject with prostate cancer, the method comprising: a) providing a first sample from the prostate from the subject suspected of comprising cancer cells; b) contacting the first sample with a first probe capable of specifically recognizing a 16p13.3 chromosome region, wherein the first probe is associated with a first label; c) contacting the first sample with a first reference probe capable of specifically recognizing a reference region of chromosome 16, wherein the first reference probe is associated with a first reference label; d) detecting the signal from the first label and from the first reference label; and e) classifying the cancer risk of the subject based on the presence or absence of a 16p13.3 gain, wherein the 16p13.3 gain is detected if the signal from the first label of the first probe is greater than the signal from the first reference label of the first reference probe. In one embodiment, the method further comprises f) providing a second sample from the prostate from the subject suspected of comprising cance cells; g) contacting the second sample with a second probe capable of specifically recognizing a 10q23.3 (PTEN) chromosome region, wherein the second probe is associated with a second label; h) contacting the second sample with a second reference probe capable of specifically recognizing a reference region of chromosome 10, wherein the second reference probe is associated with a second reference label; i) detecting the signal from the second label and from the second reference label; and j) classifying the cancer risk of the subject based on the presence or absence of a PTEN deletion, wherein the PTEN deletion is detected if the signal from the second label is less than the signal from the second reference label.

In a second aspect, the present disclosure provides a method of determining the presence or absence of aggressive prostate cancer in a subject, the method comprising: a) providing a first sample from the prostate from the subject suspected of comprising cancer cells; b) contacting the first sample with a first probe capable of specifically recognizing a 16p13.3 chromosome region, wherein the first probe is associated with a first label; c) contacting the first sample with a first reference probe capable of specifically recognizing a reference region of chromosome 16, wherein the first reference probe is associated with a first reference label; d) detecting the signal from the first label and from the first reference label; and e) characterizing the prostate cancer aggressiveness of the subject based on the presence or absence of a 16p13.3 gain, wherein the 16p13.3 gain is detected if the signal from the first label is greater than the signal from the first reference label, and the subject is characterized as having the aggressive prostate cancer in the presence of the 16p13.3 gain. In one embodiment, the method further comprises f) providing a second sample from the prostate from the subject suspected of comprising cancer calls; g) contacting the second sample with a second probe capable of specifically recognizing a 10q23.3 (PTEN) chromosome region, wherein the second probe is associated with a second label; h) contacting the second sample with a second reference probe capable of specifically recognizing a reference region of chromosome 10, wherein the second reference probe is associated with a second reference label; i) detecting the signal from the second label and from the second reference label; and j) characterizing the prostate cancer aggressiveness of the subject based on the presence or absence of a PTEN deletion, wherein the PTEN deletion is detected if the signal from the second label is less than the signal from the second reference label, and the subject is characterized as having the aggressive prostate cancer in the presence of the PTEN deletion.

In a third aspect, the present disclosure provides a method of determining recurrence-free and/or metastasis-free survival in a subject having prostate cancer, the method comprising: a) providing a first sample of the prostate from the subject suspected of comprising cancer cells; b) contacting the first sample with a first probe capable of specifically recognizing a 16p13.3 chromosome region, wherein the first probe is associated with a first label; c) contacting the first sample with a first reference probe capable of specifically recognizing a reference region of chromosome 16, wherein the first reference probe is associated with a first reference label; d) detecting the signal from the first label and from the first reference label; and e) determining the recurrence-free or metastasis-free survival of the subject based on the presence or absence of a 16p13.3 gain, wherein the 16p13.3 gain is detected if the signal from the first label of the first probe is greater than the signal from the first reference label of the first reference probe, and the subject is characterized as having a reduced the recurrence-free or metastasis-free survival in the presence of the 16p13.3 gain. In one embodiment, the method further comprises f) providing a second sample from the prostate from the subject suspected of comprising cancer cells; g) contacting the second sample with a second probe capable of specifically recognizing a 10q23.3 (PTEN) chromosome region, wherein the second probe is associated with a second label; h) contacting the second sample with a second reference probe capable of specifically recognizing a reference region of chromosome 10, wherein the second reference probe is associated with a second reference label; i) detecting the signal from the second label and from the second reference label; and j) determining the recurrence-free or metastasis-free survival of the subject based on the presence or absence of a PTEN deletion, wherein the PTEN deletion is detected if the signal from the second label is less than the signal from the second reference label, and the subject is characterized as having a reduced the recurrence-free or metastasis-free survival in the presence of the PTEN deletion.

In some embodiments, the sample is a tumor sample. In one embodiment, the first sample is the second sample. In some embodiments, the subject presents low to intermediate risk clinical features. In some embodiments, the first probe is derived from a RP11-20123 DNA clone, variants thereof, fragments thereof, or complements thereof. While in other embodiments, the first probe is capable of hybridizing to one or more genes of the 16p13.3 chromosome region comprising PKD1, RAB26, TRAF7, CASKIN1, MLST8, PGP, E4F1, DNASE1L2, ECI1, RNPS1, ABCA3, ABCA17A, CCNF, NTN3, TBC1D24, ATP6V0C, AMDHD2, CEMP1, PDPK1, variants thereof, fragments thereof, or complements thereof. In some embodiments, the first reference probe is capable of specifically recognizing a 16qh centromeric region. In one embodiment, the first reference probe is a derived from a pHuR-195 DNA clone, variants thereof, fragments thereof, or complements thereof. In some embodiments, the second probe is derived from a CTD-2557P6 DNA clone, variants thereof, fragments thereof, or complements thereof. In some embodiments, the second reference label is associated with a second reference probe capable of specifically recognizing a 10p11.1-q11.1 centromeric region. In one embodiment, the second reference probe is a CEP10 probe, variants thereof, fragments thereof, or complements thereof. In some embodiments, the probes are covalently bonded to the labels. In some embodiments, the labels are fluorescent labels. In one embodiment, the fluorescent labels are detected with fluorescent in situ hybridization (FISH) method. In some embodiments, the method comprises classifying the tumor of the subject prior to step (a). In some embodiments, the method further comprises treating the subject with an adjuvant or a neoadjuvant therapy if the subject is characterized as being associated with poor prognosis. In some embodiments, the adjuvant or neoadjuvant therapy comprises surgery, radiation therapy, hormonotherapy and/or chemotherapy. In some embodiments, the subject is a human. In some embodiments, the subject has previously had a radical prostatectomy, and in one embodiment, the sample is from a resected tissue. In some embodiments, the sample is from a biopsy.

In a fourth aspect, the present disclosure provides a method of treating a subject having prostate cancer with an adjuvant or a neoadjuvant therapy, the method comprising: a) providing a tumor sample of the prostate cancer from the subject; b) performing the method of any one of claims 1 to 26 on the sample to determine if the subject has a 16p13.3 gain and optionally a PTEN deletion; c) characterizing the prognosis of the subject; and d) if the subject has a poor prognosis, administering an adjuvant or neoadjuvant therapy to the subject. In some embodiments, the adjuvant or neoadjuvant therapy surgery, radiation therapy, hormonotherapy and/or chemotherapy.

In a fifth aspect, the present disclosure provides a kit for the assessment of a cancer prognosis in a subject suspected of having or having prostate cancer, the kit comprising: a) a first probe capable of specifically recognizing a 16p13.3 chromosome region, wherein the first probe is associated with a first label; and b) a first reference probe capable of specifically recognizing a reference region of chromosome 16, wherein the first reference probe is associated with a first reference label. In one embodiment, the kit further comprises c) a second probe capable of specifically recognizing a 10q23.3 (PTEN) chromosome region, wherein the second probe is associated with a second label; and d) a second reference probe capable of specifically recognizing a reference region of chromosome 10, wherein the second reference probe is associated with a second reference label.

### BRIEF DESCRIPTION OF THE DRAWINGS

Having thus generally described the nature of the invention, reference will now be made to the accompanying drawings and tables, showing by way of illustration, a preferred embodiment thereof, and in which:

FIG. 1A to 1D show a dual-color FISH analysis of 16p13.3 gain in FFPE prostate cancer specimens. All representative pictures were taken at X96 magnification.

FIG. 1A The arrows indicate, normal interphase nuclei with 2 orange signals (16p13.3 locus) and 2 green signals (centromere 16) in the tumor specimen with no 16p13.3 gain.

FIG. 1B The arrows indicate nuclei with 3 orange signals (16p13.3 locus) and 2 green signals (centromere 16) per nucleus, indicating a single copy 16p13.3 gain in a prostate tumor.

FIG. 1C The arrows indicate nuclei with high level of gain (>3 orange signals and 2 green signals) in a prostate tumor.

FIG. 1D The pie chart represents the proportion of RP specimens in McGill cohort, harboring 16p13.3 gain as assessed by FISH.

FIG. 2A to 2E show prognostic value of the 16p13.3 genomic gain in primary tumors of prostate cancer patients. Kaplan-Meier recurrence-free survival analysis of prostate cancer patients stratified on the basis of 16p13.3 gain status determined by FISH. Number of patients at risk at respective time points and P value (log-rank test) are indicated.

FIG. 2A All RP patients with clinical follow-up for BCR (n=238)

FIG. 2B The subgroup of patients belonging to low-intermediate risk patients with PSA≤10 (n=189).

FIG. 2C Patients with GS≤7 (C; n=222).

FIG. 2D Patients with stage T2 (n=164).

FIG. 2E Kaplan-Meier metastases-free survival analysis of all cases stratified on the basis of 16p13.3 gain status (n=257).

FIG. 3A to 3D show improved risk stratification upon combination of 16p13.3 gain and standard prognostic markers. Number of patients at risk at respective time points and P value (log-rank test) are indicated. The 16p13.3 gain status was combined with individual standard clinicopathologic variables:

FIG. 3A PSA (≤10 vs.>10 ng/mL);

FIG. 3B GS (≤GS7 vs.≥8); and

FIG. 3C pT-stage (pT2 vs. pT3) and cases with zero, one or two positive markers were compared by Kaplan-Meier analyses.

FIG. 3D The 16p13.3 gain status and all the 3 clinico-pathologic variables described above were used to stratify patients and cases with zero, one, two, three, or all four positive markers were grouped to compare BCR outcome.

FIG. 4A to 4C show 16p13.3 gain status affords additional prognostic information to the preestablished CAPRA-S score risk groups. Number of patients at risk at respective time points and P value (log-rank test) are indicated.

FIG. 4A Kaplan-Meier curves validating the prognostic significance of CAPRA-S risk groups when stratified as low risk (LR, CAPRA-S score 0-2), intermediate risk (IR, CAPRA-S score 3-5), and high risk (HR, CAPRA-S score≥6).

FIG. 4B Each CAPRA-S risk group was further subdivided on the basis of the presence (+) or absence (−) of the 16p13.3 gain.

FIG. 4C Four risk groups were derived by merging the groups with the overlapping risk of BCR from FIG. 4B as follows: LR^{+/-}; IR⁺ or HR⁻; and HR⁺; respectively.

FIGS. 5A and 5B show 16p13.3 gain status affords additional prognostic information to the pre-established CAPRA-S score risk groups in an independent dataset from Taylor et al. Number of patients at risk at respective time points and P-value (log-rank test) are indicated.

FIG. 5A Kaplan-Meier curves of CAPRA-S risk groups when stratified as Low Risk (LR, CAPRA-S score 0-2), Intermediate Risk (IR, CAPRA-S score 3-5) and High Risk (HR, CAPRA-S score≥6).

FIG. 5B The four risk groups incorporating the CAPRA-S score with the presence (+) or absence (−) of the 16p13.3 gain as follows: LR^{+/-}; IR⁺ or HR⁻; and HR⁺; respectively.

FIG. 6A to 6D show dual-color FISH analysis of PTEN (10q23) deletion in prostate cancer specimens.

FIG. 6A The arrows show normal interphase nuclei with 2 green and 2 orange signals in a PCa tumor with no PTEN deletion.

FIG. 6B The arrows show 2 green and 1 orange signals in a tumor harboring hemizygous PTEN deletion.

FIG. 6C The arrow shows 2 green and 0 orange signals in a homozygous PTEN-deleted case. FISH analysis.

FIG. 6D The pie chart represents detected hemizygous in 80/287 (28%), homozygous in 17/287 (6%), and no PTEN deletion in 189/287 (66%) of the primary radical prostatectomy samples on the McGill urology tissue microarray (n=287).

FIG. 7A to 7H show prognostic value of the PTEN genomic deletion in prostate tumors. Kaplan-Meier recurrence-free survival analysis of patients stratified on the basis of PTEN deletion status determined by FISH. Censored data (tick marks), number of patients at risk at respective time points, and P-value (log-rank test) are indicated. The results are shown in:

FIG. 7A All radical prostatectomy patients with clinical follow-up for biochemical recurrence;

FIG. 7B Gleason grade group 1-2 (≤3+4),

FIG. 7C Gleason grade group 1-2 (≤3+4), stage pT2 and prostate-specific antigen 10 patients;

FIG. 7D Gleason grade group 2 (3+4);

FIG. 7E Gleason grade group 2 (3+4), stage pT2 and prostate-specific antigen 10 patients;

FIG. 7F low CAPRA-S score (0-2) risk patients; and

FIG. 7G intermediate CAPRA-S score (Klotz et al., 2014; Chang et al., 2012; Epstein et al., 2012) risk patients.

FIG. 7H Survival analysis based on PTEN deletion status shows worse metastases-free survival.

FIG. 8A to 8D show improved risk stratification upon combination of 16p13.3 gain and PTEN genomic deletion. Censored data (tick marks), number of patients at risk at respective time points, and P-value (log-rank test) are indicated. Kaplan-Meier recurrence-free survival analysis of prostate cancer patients stratified on the basis of FIG. 8A PTEN deletion status in cases harboring no 16p13 gain;

FIG. 8B 16p13 gain status in cases harboring no PTEN deletion; and

FIG. 8C in patients with none, either or both genomic alterations.

FIG. 8D Harrel C-index is improved when combining 16p13 gain, PTEN deletion and CAPRA-S score risk group, compared to individual markers or either of the three.

### DETAILED DESCRIPTION

Genomic profiling studies have highlighted disease heterogeneity, and in particular DNA copy number alterations (CNA) are common in cancer and have been associated with molecular subtypes of prostate cancer, supporting the existence of alternative parallel pathways of tumorigenesis.

The terms “cancer” and “cancerous” refer to or describe the physiological condition in mammals that is typically characterized by unregulated cell growth. Examples of cancer include but are not limited to, carcinoma, lymphoma, blastoma, sarcoma, melanoma, and leukemia. More particular examples of such cancers include squamous cell cancer, small-cell lung cancer, non-small-cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma, and various types of head and neck cancer. As used herein, “prostate cancer” refers to cancer in the prostate gland in the male reproductive system, and may include cancer in the surrounding lymph nodes as well as metastatic tumors. As used herein, “prostatectomy” refers to the surgical removal of all or part of the prostate gland and surrounding tissue.

By “nucleic acid” is meant to include any DNA or RNA, for example, chromosomal, mitochondrial, viral and/or bacterial nucleic acid present in tissue sample. The term “nucleic acid” encompasses either or both strands of a double stranded nucleic acid molecule and includes any fragment or portion of an intact nucleic acid molecule.

By “gene” is meant any nucleic acid sequence or portion thereof with a functional role in encoding or transcribing an RNA (rRNA, tRNA, or mRNA, the latter capable of 15 translation as a protein) or regulating other gene expression. The gene may consist of all the nucleic acids responsible for encoding a functional protein or only a portion of the nucleic acids responsible for encoding or expressing a protein. The nucleic acid sequence may contain a genetic abnormality within exons, introns, initiation or termination regions, promoter sequences, other regulatory sequences or unique adjacent regions to the gene.

In some embodiments, methods of prognosing a subject with prostate cancer are provided. As used herein, “prognosis” refers to the prediction of the likely or expected development of a disease, such as cancer, and includes a prediction of whether the signs and symptoms will improve, worsen, or remain stable over time; life expectancy; presence and number of metastasis; life quality expectations; potential complications; and likelihood of survival. A poor prognosis is associated with the development of the disease, the worsening of the symptoms, a reduction in life expectancy; the presence or development of metastasis, the worsening of life quality, the presence of complication, a more aggressive cancer, and/or the reduction of survival (including recurrence-free and survival-free).

In some embodiments, methods of determining the presence or absence of aggressive prostate cancer in a subject is provided. As used herein, “aggressive cancer” refers to tumor that forms, grows, or spreads quickly, and/or fails to respond to therapy (medication or radiation or both).

In some embodiments, methods of predicting recurrence-free and/or metastasis-free survival in a subject having prostate cancer. The term “recurrence-free survival” refers to the length of time after primary treatment for a cancer ends that the patient survives without any signs or symptoms (for example, the rise of PSA level) of that cancer. For example, the recurrence-free survival can be the time from primary treatment to the first radiologically detected metastasis. The term “metastases-free survival” refers to time from the time interval from prostate-specific antigen (PSA) recurrence to first radiographically detected metastasis.

In some embodiments, the subjects have not been previously diagnosed with prostate cancer. In such embodiments, the subjects may have elevated PSA levels and the methods can be used to determine if further investigation or treatment is needed or if only active surveillance is warranted. In some alternative embodiments, the subjects may have been previously diagnosed with prostate cancer. In such embodiments, some of the subjects may not have received primary treatment and the methods can be used to determine if therapy is warranted or active surveillance is preferred. Alternatively, the subjects may have received primary treatment, such as radical prostatectomy or radiation therapy and the methods can be used to determine if additional therapy is warranted (hormonotherapy and/or chemotherapy for example) or if an active surveillance is preferred.

In some embodiments, subjects have prostate-specific antigen (PSA) which are detected in serum samples. In some embodiments, the methods of the present disclosure include determining the level of serum PSA of subjects and taking this information to determine the cancer risk, the predisposition to aggressive cancer and/or to determine survival likelihood. PSA level can be used to stratify the individuals according to the methods disclosed herein. In some embodiments, the subjects can be classified as low-intermediate risk using standard clinicopathologic prognostic markers such as PSA. In some embodiments, the subjects with prostate cancer are classified as high risk using standard clinicopathologic prognostic markers such as PSA.

The tumors of subjects suspected of having or having been diagnosed with prostate cancer can be classified according to the Gleason score. In some embodiments, the methods of the present disclosure include determining the Gleason score of the tumor of the subjects and taking this information to determine the cancer risk, predisposition to aggressive cancer and/or to determine likelihood of survival. Grading a tumor using the Gleason score is difficult. Usually, individuals scoring 7 or higher are treated (with surgery or radiotherapy) whereas individuals scoring 6 or lower are actively monitored. The methods described herein may be particularly useful if the subject scores a 6 or lower on the Gleason score as it may provide important information to determine if a more aggressive therapy is warranted or if active surveillance is more appropriate.

The methods described herein can be repeated in time (monthly, yearly for example) to determine if the prostate cancer evolves or remains stable.

By “sample”, it is meant a collection of cells from a prostate suspected of being cancerous. The sample can be, for example, a biopsy or a resected tissue obtained with surgery. The sample can be derived from epithelium tissue; connective tissues, including blood vessels, bone and cartilage; muscle tissue; or nerve tissue. In one embodiment, the tumor sample is obtained from prostate gland tissue. The source of the sample maybe solid tissue as from a fresh, frozen and/or preserved organ or tissue sample or biopsy or aspirate; blood or any blood constituents; bodily fluids such as cerebral spinal fluid, amniotic fluid, peritoneal fluid, or interstitial fluid of the subject. The sample may also be primary or cultured cells. The sample may contain compounds which are not naturally intermixed with the tissue in nature such as preservatives, anticoagulants, buffers, fixatives, nutrients, antibiotics, or the like. By “tumor sample”, it is meant a collection of cells obtained from a cancerous tissue (such as a cancerous prostate) of a subject or patient, in which some or all of the collection of cells exhibit unregulated cancer cell growth. The tumor sample can be obtained from the primary tumor or a tumor metastasis or both.

For the purposes herein a “section” of a tumor sample is meant a single part or piece of a tumor sample, e.g., a thin slice of tissue or cells cut from a tumor sample. It is understood that multiple sections of tumor samples may be taken and subjected to analysis according to the present disclosure, provided that it is understood that the present disclosure comprises a method whereby the same section of tissue sample may be analyzed at both morphological and molecular levels, or may be analyzed with respect to both protein and nucleic acid content. In an embodiment, the tumor sample is modified prior to the detection of the genetic markers. For example, the tumor sample can be fixed prior to the detection of the genetic markers.

As presented in the examples, it was shown that the 16p13.3 genomic gain is a predictor of poor clinical outcome in prostate cancer. Accordingly, detection of a 16p13.3 genomic gain can aid in better risk stratifying subjects when combined with standard clinicopathologic prognostic markers. As presented in the examples, it was also shown that the PTEN genomic deletion is a strong independent predictor of poor clinical outcome, including in low-intermediate risk patients and showed that the combination of the PTEN deletion and the 16p13.3 gain status improved patient risk stratification.

As used herein, “genomic gain” or “gain” is an amplification of a section of DNA in the context of cancer where a tumor exhibits copy number gain at different loci when compared to a normal/healthy sample. While “genomic deletion” or “deletion” refers to where a tumor exhibits copy number loss at different loci when compared to a normal/healthy sample. Both of these terms are referred to as copy number alterations (CNAs).

The present disclosure also provides methods of treating a subject having prostate cancer is provided, with an adjuvant or neoadjuvant therapy. “Treatment” refers to both therapeutic treatment and prophylactic or preventative measures. Those in need of treatment include those already with the disorder as well as those in which the disorder is to be prevented. The term adjuvant therapy refers to additional cancer treatment given after the primary treatment to lower the risk that the cancer will come back. Adjuvant therapy may include chemotherapy, radiation therapy, hormone therapy, targeted therapy, or biological therapy. The term neoadjuvant therapy refers to a treatment given as a first step to shrink a tumor before the main treatment, which is usually surgery, is given. Examples of neoadjuvant therapy also include chemotherapy, radiation therapy, and hormonal therapy.

### Probes for Determining Copy Number Alterations.

In the context of the present disclosure, samples (which can be tumor samples) obtained from a tissue such as the prostate (which can be from a tumor located in the prostate or a metastasis which originated from the prostate) of a subject are analyzed for chromosomal abnormalities, including DNA copy number alterations (CNA). In some embodiments, the samples are fixed to preserve the samples from decay due to autolysis or putrefaction. In some embodiments, samples are fixed using chemical fixatives, such as formaldehyde, glutaraldehyde, alcohols, mercurials, or picrates. In one embodiment, the tumor sample are formalin-fixed paraffin-embedded (FFPE). In some embodiments, the tumor samples are sliced into tissue sections for analysis.

In some embodiments, a DNA CNA is detected using labeled probes, preferably fluorescent labeled probes and detected using florescence microscopy. In some embodiments, labels are provided separately from target specific probes and binds to the probes for specific detection of a target DNA sequence. In other embodiments, labels are bonded to target specific probes for specific detection of a target DNA sequence.

As used herein, the term “probe” or “hybridization probe” is a fragment of DNA or RNA of variable length that specifically hybridizes to a genetic target to detect the presence of a target nucleotide sequence that is complementary to the sequence in the probe. The probe can be labeled. The probe is at least 10, 100 or 1 000 nucleotides long and, in some embodiments, can span several thousand nucleotides. The probe can span the genetic target or can be smaller than the genetic target. A collection of probes overlapping or non-overlapping probes can also be used. As used herein, the term “hybridization” refers to annealing of a single-stranded nucleic acid to a complementary nucleic acid. A probe which is capable of hybridizing to a complementary target sequence does not need to be 100% complementary to the complementary sequence. In some embodiments, the probe is at least 50, 55, 60, 65, 70, 75, 80, 85, 90, 95, 96, 97, 98 or 99% complementary to the complementary target sequence. Preferably such hybridization between the probe and its complementary nucleic acid sequence is specific, i.e., it occurs under high stringency conditions.

The term “label” when used herein refers to a compound or composition which is bonded or fused directly or indirectly to a reagent, or binds to a reagent, such as a nucleic acid probe or an antibody and facilitates detection of the reagent to which it is conjugated or fused. The label may itself be detectable (e.g., radioisotope labels or fluorescent labels) or, in the case of an enzymatic label, may catalyze chemical alteration of a substrate compound or composition which is detectable. A hapten or epitope that is immunospecifically bound by an antibody can also serve as a label. In preferred embodiments, the label is a fluorescent label. The probe can include one or several labels. When a method is practiced with more than one probe, each different probe type is labelled with a different label to allow discriminating between the different probe types.

The term “labeled probe” or “probe associated with a label” refers to a probe comprising (1) a nucleic acid having a sequence rendering it capable of hybridizing with a target nucleic acid sequence that is bonded or fused directly or indirectly, or bound with (2) a label. Preferably such hybridization is specific, i.e., it occurs under high stringency conditions.

Probes should have sufficient complementarity to the target nucleic acid sequence of interest so that stable and specific binding occurs between the target nucleic acid sequence and the probe. The degree of homology/identity required for stable hybridization varies with the stringency of the hybridization medium and/or wash medium. Preferably, completely homologous probes are employed in the present disclosure, but persons of skill in the art will readily appreciate that probes exhibiting lesser but sufficient homology can be used in the present disclosure (see e.g., Sambrook, J., et al., Molecular Cloning A Laboratmy Manual, Cold Spring Harbor Press, (1989)).

Probes may also be generated and chosen by several means including, but not limited to, mapping by in situ hybridization, somatic cell hybrid panels, or spot blots of sorted chromosomes; chromosomal linkage analysis; or cloned and isolated from sorted chromosome libraries from human cell lines or somatic cell hybrids with human chromosomes, radiation somatic cell hybrids, microdissection of a chromosome region, or from yeast artificial chromosomes (YACs) or bacterial artificial chromosomes (BAC) identified by PCR primers specific for a unique chromosome locus or other suitable means like an adjacent YAC or BAC clone. Probes may be genomic DNA, eDNA, or RNA cloned in a plasmid, phage, cosmid, YAC, BAC, viral vector, or any other suitable vector. Probes may be cloned or synthesized chemically by conventional methods. When cloned, the isolated probe nucleic acid fragments are typically inserted into a vector, such as lambda phage, pBR322, M13, or vectors containing the SP6 or T7 promoter and cloned as a library in a bacterial host (see, e.g., Sambrook, supra).

In one embodiment, the labeled probes are fluorescent and can be used, for example, in a method of fluorescent in situ hybridization (FISH). In such embodiments, the probes specifically bind to the target sequence of a chromosome with a high degree of sequence complementarity. In situ hybridization is generally carried out on cells or tissue sections fixed to slides. In situ hybridization may be performed by several conventional methodologies (see, e.g., Leitch et al., In Situ Hybridization: A Practical Guide, Oxford BIOS Scientific Publishers, Micropscopy Handbooks v. 27 (1994)). In one in situ procedure, fluorescent dyes (such as fluorescein isothiocyanate (FITC) which fluoresces green when excited by an Argon ion laser) are used to label a nucleic acid sequence probe that is complementary to a target nucleotide sequence in the cell. Each cell containing the target nucleotide sequence will bind the labeled probe producing a fluorescent signal upon exposure, of the cells to a light source of a wavelength appropriate for excitation of the specific fluorochrome used. FISH analysis can be used in conjunction with other assays, including without limitation morphological staining (of serial sections or the same section; see PCT Publication No. WO 00/20641).

Various degrees of hybridization stringency can be employed to allow the binding/washing of the probe to its complementary target sequence. As the hybridization conditions become more stringent, a greater degree of complementarity is required between the probe and target to form and maintain a stable duplex. Stringency is increased by raising temperature, lowering salt concentration, or raising formamide concentration. Adding dextran sulfate or raising its concentration may also increase the effective concentration of labeled probe to increase the rate of hybridization and ultimate signal intensity. After hybridization, slides are washed in a solution generally containing reagents similar to those found in the hybridization solution with washing time varying from minutes to hours depending on required stringency. Longer or more stringent washes typically lower nonspecific background but run the risk of decreasing overall sensitivity.

Probes used in the FISH assay may be either RNA or DNA oligonucleotides or polynucleotides and may contain not only naturally occurring nucleotides but their analogs like digoxygenin dCTP, biotin dcTP 7-azaguanosine, azidothymidine, inosine, or uridine. Other useful probes include peptide probes and analogues thereof, branched gene DNA, peptidometics, peptide nucleic acid (PNA), and/or antibodies.

Probes are preferably labeled with a fluorophor as the fluorescent label. Examples of fluorophores include, but are not limited to, rare earth chelates (europium chelates), Texas Red, rhodamine, fluorescein, dansyl, Lissamine, umbelliferone, phycocrytherin, phycocyanin, or commercially available fluorophors such Spectrum Orange7 and Spectrum Green7, and/or derivatives of any one or more of the above. Multiple probes used in the assay may be labeled with more than one distinguishable fluorescent or pigment color. These color differences provide a means to identify the hybridization positions of specific probes. Moreover, probes that are not separated spatially can be identified by a different color light or pigment resulting from mixing two other colors (e.g., lightred+green=yellow), pigment (e.g., blue+yellow=green), or by using a filter set that passes only one color at a time.

Probes can be labeled directly or indirectly with the fluorescent label, utilizing conventional methodology. Additional probes and colors may be added to refine and extend this general procedure to include more genetic abnormalities or serve as internal controls. In one embodiment, FISH probes are directly labeled with the fluorescent label, or is covalently bonded to a fluorescent label.

Probes can have 10, 20, 30, 40, 50, 60, 70, 80, 90, 100, 200, 300, 400, 500 nucleic acid bases or more in length.

After processing for FISH, the slides may be analyzed by standard techniques of fluorescence microscopy (see, e.g., Ploem and Tanke, Introduction to Fluorescence Microscopy, 15 Oxford University Press: New York (1987)). Briefly, each slide is observed using a microscope equipped with appropriate excitation filters, dichromic, and barrier filters. Filters are chosen based on the excitation and emission spectra of the fluorochromes used. Photographs of the slides maybe taken with the length of time of film exposure depending on the fluorescent label used, the signal intensity and the filter chosen. For FISH analysis the physical loci of the cells of interest determined in the morphological analysis are recalled and visually conformed as being the appropriate area for FISH quantification. In some embodiments, tumor samples treated with fluorescent labeled probes are analyzed sample by sample, section by section, or on a cell by cell basis.

### 16p13.3 Gain Detection

As used herein the term “16p13.3” refers to a specific chromosomic region of chromosome 16 in humans that includes both non-coding and coding sequences. The chromosomal region 16p13.3 includes, but is not limited to genes such as: GFER, NTHL1, TSC2, PKD1, RAB26, TRAF7, CASKIN1, MLST8, PGP, E4F1, DNASE1L2, ECI1, RNPS1, ABCA3, ABCA17P, CCNF, NTN3, TBC1D24, ATP6V0C, AMDHD2, CEMP1, PDPK1, KCTD5, PRSS27, and SRRM2. As usd in the context of the present disclosure, a 16p13.3 genomic gain includes a DNA copy number gain of one or more of the chromosomal region which can include the above listed genes. In some embodiments, a 16p13.3 genomic gain involves a DNA copy number gain of one or more of PKD1, RAB26, TRAF7, CASKIN1, MLST8, PGP, E4F1, DNASE1L2, ECI1, RNPS1, ABCA3, ABCA17P, CCNF, NTN3, TBC1D24, ATP6V0C, AMDHD2 or CEMP1. In one embodiment, a 16p13.3 genomic gain involves a DNA copy number gain of PDPK1. In some embodiments, the gain is more than one and correspond to two, three, four or more amplifications.

The focal 16p13.3 genomic gain was previously mapped in primary prostate tumors and identified PDPK1 encoding 3-phosphoinositide-dependent protein kinase-1 (PDK1) as a likely driver of the gain with functional impact on prostate cancer cell migration (Choucair et al., 2012). Encoded by PDPK1, PDK1 phosphorylates and activates the AGC kinase members regulated by phosphatidylinositol 3-kinase, including AKT. In addition to its kinase activity on AKT, it has been shown that PDK1 also has an important role in in vitro prostate cancer cell migration (Choucair et al., 2012).

In some embodiments, a 16p13.3 genomic gain comprises a single copy gain of the one or more genes from the 16p13.3 region of chromosome 16. In some embodiments, a 16p13.3 genomic gain comprises two copy gain, three copy gain, four copy gain or more of the one or more genes from the 16p13.3 region of chromosome 16.

To detect a 16p13.3 genomic gain, methods provided herein comprise contacting a sample with a first 16p13.3 probe capable of specifically recognizing a 16p13.3 chromosome region, wherein the first probe is associated with a first label; and detecting the signal from the first label of the first probe. The sample is also contacted with a first reference probe comprising a first reference label to specifically bind to and detect a reference region in chromosome 16. The reference region can be any region of chromosome 16 that is not susceptible of being gained or deleted in the sample. Then, the signal from the first label is compared relative to the signal from a first reference label. A 16p13.3 gain is detected if the incident of signals from the first label of the first 16p13.3 probe is greater than the incident of signals from the first reference label probe. Adjustments can be made to detect a 16p13.3 gain, for example when the incident of signals from the first label is at least twice, thrice or more higher than the incidents of signal from the first reference label.

In some embodiments, the first 16p13.3 probe hybridizes with a 16p13.3 chromosome region of chromosome 16. This hybridization can be observed in the coding as well as in the non-coding sequences of the 16p13.3 chromosome regions. In some embodiments, the first 16p13.3 probe hybridizes with a chromosome region encoding for one or more of the genes from the 16p13.3 region of chromosome 16. In one embodiment, the first 16p13.3 probe hybridizes with a chromosome region encoding one or more of the genes: PKD1, RAB26, TRAF7, CASKIN1, GBL, PGP, E4F1, DNASE1L2, DCI, RNPS1, ABCA3, ABCA17, CCNF, NTN3, TBC1D24, KIAA1171, ATP6V0C, AMDHD2, CEMP1, or PDPK1. In some embodiments, the first 16p13.3 probe comprises a nucleotide sequence that is complementary to one or more of the fragments from the 16p13.3 region of chromosome 16, which can include, for example, one or more of the genes listed above. In one embodiment, the first 16p13.3 probe is derived from a BAC clone, such as, for example, a RP11-20123 BAC clone.

In the context of the present disclosure, the first 16p13.3 probe can be a variant probe which comprises a nucleic sequence that is complementary to or hybridizes with a chromosome region encoding a 16p13.3 region. A variant probe comprises at least one nucleotide difference (substitution, addition, or deletion) when compared the native probe. A variant includes an allele variant of a gene (dominant or recessive). In an embodiment, the first 16p13.3 variant probe and has at least 50%, 60%, 70%, 80%, 85%, 90%, 95%, 96%, 97%, 98% or 99% identity to the native probe and still be able to hybridize specifically to the 16p13.3 region. The term “percent identity”, as known in the art, is a relationship between two or more polypeptide sequences, as determined by comparing the sequences. The level of identity can be determined conventionally using known computer programs. Identity can be readily calculated by known methods, including but not limited to those described in: Computational Molecular Biology (Lesk, A. M., ed.) Oxford University Press, N Y (1988); Biocomputing: Informatics and Genome Projects (Smith, D. W., ed.) Academic Press, N Y (1993); Computer Analysis of Sequence Data, Part I (Griffin, A. M., and Griffin, H. G., eds.) Humana Press, N J (1994); Sequence Analysis in Molecular Biology (von Heinje, G., ed.) Academic Press (1987); and Sequence Analysis Primer (Gribskov, M. and Devereux, J., eds.) Stockton Press, NY (1991). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR Inc., Madison, Wis.). Multiple alignments of the sequences disclosed herein were performed using the Clustal method of alignment (Higgins and Sharp (1989) CABIOS. 5:151-153) with the default parameters (GAP PENALTY=10, GAP LENGTH PEN ALT Y=10). Default parameters for pairwise alignments using the Clustal method were KTUPLB 1, GAP PENALTY=3, WINDOW=5 and DIAGONALS SAVED=5.

The present disclosure also provides first 16p13.3 probe fragments comprising a nucleic acid sequence that is complementary to the nucleotide sequences of one or more of the genes from the 16p13.3 region of chromosome 16 and hybridizes to a chromosome region encoding a portion of the one or more of the genes from the 16p13.3 region. A fragment sequence comprises at least one less nucleotide when compared to the full-length nucleic acid sequence of the native probe. In an embodiment, the first 16p13.3 probes has a nucleic acid sequence that exhibits or has at least 30%, 40%, 50%, 60%, 70%, 80%, 85%, 90%, 95%, 96%, 97%, 98% or 99% identity to the full-length nucleic acid sequence of the native probe. The fragment sequence can be, for example, a truncation of the full-length sequence of the native probe. Alternatively or in combination, the fragment sequence can be generated from removing one or more internal nucleotides to the native probe. In an embodiment, the first 16p13.3 probes has a nucleic acid sequence that is complementary to at least 100, 150, 200, 250, 300, 350, 400, 450, 500 or more consecutive nucleotides of the one or more of the native probe.

The first reference probe is specific for a reference location on chromosome 16 and, in some embodiments, is capable of specifically recognizing a 16qh centromeric region. In one embodiment, the first reference probe is derived from a pHuR-195 BAC clone, variants thereof, fragments thereof or complements thereof.

In the context of the present disclosure, the first reference probe comprises a variant nucleic sequence that is complementary to or hybridizes with a 16qh centromeric region including variants of one or more of the genes from the 16qh centromeric region of chromosome 16. A variant probe comprises at least one nucleotide difference (substitution, addition, or deletion) when compared to the native probe. A variant can include, for example, an allele variant of a gene in the chromosomal region (dominant or recessive). In an embodiment, the first reference variant probe hybridizes with a 16qh centromeric region of chromosome 16 and has at least 50%, 60%, 70%, 80%, 85%, 90%, 95%, 96%, 97%, 98% or 99% identity to the nucleic acid sequence of the native probe. The term “percent identity”, as known in the art, is a relationship between two or more polypeptide sequences, as determined by comparing the sequences. The level of identity can be determined conventionally using known computer programs. Identity can be readily calculated by known methods, including but not limited to those described in: Computational Molecular Biology (Lesk, A. M., ed.) Oxford University Press, N Y (1988); Biocomputing: Informatics and Genome Projects (Smith, D. W., ed.) Academic Press, N Y (1993); Computer Analysis of Sequence Data, Part I (Griffin, A. M., and Griffin, H. G., eds.) Humana Press, N J (1994); Sequence Analysis in Molecular Biology (von Heinje, G., ed.) Academic Press (1987); and Sequence Analysis Primer (Gribskov, M. and Devereux, J., eds.) Stockton Press, NY (1991). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR Inc., Madison, Wis.). Multiple alignments of the sequences disclosed herein were performed using the Clustal method of alignment (Higgins and Sharp (1989) CABIOS. 5:151-153) with the default parameters (GAP PENALTY=10, GAP LENGTH PEN ALT Y=10). Default parameters for pairwise alignments using the Clustal method were KTUPLB 1, GAP PENALTY=3, WINDOW=5 and DIAGONALS SAVED=5.

The present disclosure also provides a first reference probe fragment comprising a nucleic acid sequence that is complementary to the 16qh centromeric region of chromosome 16 and hybridizes to a chromosome region encoding a portion of the one or more of the genes from the 16qh centromeric region. A fragment sequence comprises at least one less nucleotide when compared to the full-length nucleic acid sequence of the native probe or variant thereof. In an embodiment, the first reference fragment probe has a nucleic acid sequence that exhibits or has at least 30%, 40%, 50%, 60%, 70%, 80%, 85%, 90%, 95%, 96%, 97%, 98% or 99% identity to the full-length nucleic acid sequence of the native probe. The fragment sequence can be, for example, a truncation of the full-length sequence of the native probe. Alternatively or in combination, the fragment sequence can be generated from removing one or more internal nucleotides form the native probe. In an embodiment, the first reference fragment probe has a nucleic acid sequence that is complementary to at least 100, 150, 200, 250, 300, 350, 400, 450, 500 or more consecutive nucleotides of the one or more of the native probe.

### PTEN Deletion Detection

The phosphatidylinositol 3-kinase (PI3K)/AKT signal transduction pathway contributes to cancer growth and survival, and is activated in a broad range of human malignancies including prostate cancer. The phosphatase and tensin homologue deleted on chromosome 10 (PTEN) is a tumor suppressor gene on 10q23.3 locus that acts by negatively regulating the PI3K/AKT pathway. PTEN genomic deletion has been detected in human tissues representing all stages of prostate cancer development and progression including High Grade Prostatic Intraepithelial Neoplasia (HGPIN), primary PCa and at higher frequency in metastatic prostate cancer and castrate resistant prostate cancer.

To detect a PTEN genomic deletion, methods are provided comprising contacting a sample with a second (PTEN) probe capable of specifically recognizing a 10q23.3 (PTEN) chromosome region, wherein the second probe is associated with a second label; and detecting the signal from the second label. The sample is also contacted with a second reference probe comprising a second reference probe comprising a second reference label to specifically bind to and detect a reference region in chromosome 10. The reference region can be any region of chromosome 10 that is not susceptible of being gained or deleted in the sample. Then, the signal of the second label is compared to the signal of the second reference label. A PTEN deletion is detected if the incidents of signals from the second label of the second probe is less than the incidents of signal from the second reference label from the second reference probe. Adjustments can be made to detect a PTEN deletion, for example, when the incident of signals from the first label is at least twice, thrice or more lower than the incidents of signal from the first reference label.

In some embodiments, the second PTEN probe hybridizes with a 10q23.3 region of chromosome 10. In some embodiments, the second PTEN probe hybridizes with a chromosome region encoding the PTEN gene. In some embodiments, the second PTEN probe comprises a nucleotide sequence that is complementary to the PTEN gene. In one embodiment, the second PTEN probe is derived from a trans a BAC clone, such as, for example, a CTD-2557P6 BAC clone, variants thereof, fragments thereof, or complements thereof.

In some embodiments, a sample comprises cells having an homozygous PTEN deletion where both copies of PTEN are deleted. In other embodiments, a sample comprises cells having an heterozygous PTEN deletion where one copy of PTEN is deleted.

In the context of the present disclosure, the second PTEN probe can be a variant which comprises a nucleic sequence that is complementary to or hybridizes the 10q23.3 region. A variant of a probe comprises at least one nucleotide difference (substitution, addition, or deletion) when compared to the native probe. A variant can include an allele variant of a gene in the 10q23.3 region (dominant or recessive). In an embodiment, the second PTEN variant probe hybridizes with the 10q23.3 region of chromosome 10 and has at least 50%, 60%, 70%, 80%, 85%, 90%, 95%, 96%, 97%, 98% or 99% identity to the nucleic acid sequence of the native probe. The term “percent identity”, as known in the art, is a relationship between two or more polypeptide sequences, as determined by comparing the sequences. The level of identity can be determined conventionally using known computer programs. Identity can be readily calculated by known methods, including but not limited to those described in: Computational Molecular Biology (Lesk, A. M., ed.) Oxford University Press, N Y (1988); Biocomputing: Informatics and Genome Projects (Smith, D. W., ed.) Academic Press, N Y (1993); Computer Analysis of Sequence Data, Part I (Griffin, A. M., and Griffin, H. G., eds.) Humana Press, N J (1994); Sequence Analysis in Molecular Biology (von Heinje, G., ed.) Academic Press (1987); and Sequence Analysis Primer (Gribskov, M. and Devereux, J., eds.) Stockton Press, NY (1991). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR Inc., Madison, Wis.). Multiple alignments of the sequences disclosed herein were performed using the Clustal method of alignment (Higgins and Sharp (1989) CABIOS. 5:151-153) with the default parameters (GAP PENALTY=10, GAP LENGTH PEN ALT Y=10). Default parameters for pairwise alignments using the Clustal method were KTUPLB 1, GAP PENALTY=3, WINDOW=5 and DIAGONALS SAVED=5.

The present disclosure also provides second PTEN probe fragments comprising a nucleic acid sequence that is complementary the 10q23.3 region of chromosome 10 and hybridizes to a chromosome region encoding a portion of the PTEN gene from the 10q23.3 region. A fragment sequence comprises at least one less nucleotide when compared to the full-length nucleic acid sequence of the native probe. In an embodiment, the second PTEN variant probe has a nucleic acid sequence that exhibits or has at least 30%, 40%, 50%, 60%, 70%, 80%, 85%, 90%, 95%, 96%, 97%, 98% or 99% identity to the full-length nucleic acid sequence of the native probe. The fragment sequence can be, for example, a truncation of the full-length sequence of the native probe. Alternatively or in combination, the fragment sequence can be generated from removing one or more internal nucleotides from the native probe. In an embodiment, a second PTEN probe has a nucleic acid sequence that is complementary to at least 100, 150, 200, 250, 300, 350, 400, 450, 500 or more consecutive amino acids of the native probe or the variant second probe.

The second reference probe is specific for a reference location on chromosome 10 and, in some embodiments, is capable of specifically recognizing a 10p11.1-q11.1 centromeric region. In one embodiment, the second reference chromosome 10 probe is derived from a CEP10 probe, a variant thereof, a fragment thereof or a complement thereof.

In the context of the present disclosure, the second reference probe comprises variant nucleic sequence that is complementary to or hybridizes with a 10p11.1-q11.1 centromeric region. A variant of a probe comprises at least one nucleotide difference (substitution, addition, or deletion) when compared to the native probe. A variant can include an allele variant of a gene present in the 10p11.1-q11.1 centromeric region (dominant or recessive). In an embodiment, the second reference variant probe hybridizes with the 10p11.1-q11.1 centromeric region of chromosome 10 and has at least 50%, 60%, 70%, 80%, 85%, 90%, 95%, 96%, 97%, 98% or 99% identity to the nucleic acid sequence of the native probe. The term “percent identity”, as known in the art, is a relationship between two or more polypeptide sequences, as determined by comparing the sequences. The level of identity can be determined conventionally using known computer programs. Identity can be readily calculated by known methods, including but not limited to those described in: Computational Molecular Biology (Lesk, A. M., ed.) Oxford University Press, N Y (1988); Biocomputing: Informatics and Genome Projects (Smith, D. W., ed.) Academic Press, N Y (1993); Computer Analysis of Sequence Data, Part I (Griffin, A. M., and Griffin, H. G., eds.) Humana Press, N J (1994); Sequence Analysis in Molecular Biology (von Heinje, G., ed.) Academic Press (1987); and Sequence Analysis Primer (Gribskov, M. and Devereux, J., eds.) Stockton Press, NY (1991). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR Inc., Madison, Wis.). Multiple alignments of the sequences disclosed herein were performed using the Clustal method of alignment (Higgins and Sharp (1989) CABIOS. 5:151-153) with the default parameters (GAP PENALTY=10, GAP LENGTH PEN ALT Y=10). Default parameters for pairwise alignments using the Clustal method were KTUPLB 1, GAP PENALTY=3, WINDOW=5 and DIAGONALS SAVED=5.

The present disclosure also provides second reference probe fragments comprising a nucleic acid sequence that is complementary to the 10p11.1-q11.1 centromeric region of chromosome 10 and hybridizes to the 10p11.1-q11.1 centromeric region. A fragment sequence comprises at least one less nucleotide when compared to the full-length nucleic acid sequence of the native probe. In an embodiment, the second reference probe fragment has a nucleic acid sequence that exhibits or has at least 30%, 40%, 50%, 60%, 70%, 80%, 85%, 90%, 95%, 96%, 97%, 98% or 99% identity to the full-length nucleic acid sequence of the native probe and hybridizes to the 10p11.1-q11.1 centromeric region of chromosome 10, or complements thereof. The fragment sequence can be, for example, a truncation of the full-length sequence of the native probe or variant. Alternatively or in combination, the fragment sequence can be generated from removing one or more internal nucleotides from the native probe. In an embodiment, a second reference probe fragment has a nucleic acid sequence that is complementary to at least 100, 150, 200, 250, 300, 350, 400, 450, 500 or more consecutive amino acids of the native probe.

### Identifying and Classifying Cancer Risk

In some embodiments, methods of prognosing a subject with prostate cancer involve classifying the cancer risk of the subject based on the presence or absence of 16p13.3 gain. As shown herein, the presence of a 16p13.3 gain is associated with the presence of prostate cancer and generally a poor prognosis. In one embodiment, classifying the cancer risk of the subject is based on the presence or absence of 16p13.3 gain in combination with a PTEN deletion. As shown herein, the presence of a 16p13.3 gain and a PTEN deletion is associated with the presence of prostate cancer and generally a poor prognosis. The detection of the 16p13.3 gain and of the PTEN deletion can be done, for example on different samples from the subject (for example different tissue sections of a sample) or can be multiplexed on a single sample from the subject.

In some embodiments, methods of determining the presence or absence of aggressive prostate cancer in a subject involve characterizing the tumor sample based on the presence or absence of 16p13.3 gain, and the presence of 16p13.3 gain is associated with aggressive prostate cancer. In one embodiment, characterizing the tumor sample is based on the presence or absence of 16p13.3 gain and PTEN deletion, and the presence of 16p13.3 gain and PTEN deletion is associated with aggressive prostate cancer.

In some embodiments, methods of predicting recurrence-free and/or metastasis-free survival in a subject having prostate cancer involve characterizing if the subject has 16p13.3 gain, and having 16p13.3 gain is associated with a reduction of recurrence-free and/or metastases-free survival. In one embodiment, a method of predicting recurrence-free and/or metastasis-free survival in a subject having prostate cancer involves characterizing if the subject has 16p13.3 gain and PTEN deletion, where having 16p13.3 gain and PTEN deletion is associated with a reduction in the recurrence-free and/or metastases-free survival.

The method of the present disclosure can include classifying the subjects into risk groups based on clinical features or Gleason grade or CAPRA-S scores. In some embodiments, subjects are classified into low to intermediate risk groups. In some embodiments, the subjects are classified into high risk groups. The present disclosure also provides charactering the subject as being associated with higher cancer risk, or a higher cancer risk/poor prognosis relative to the assigned risked group based on clinical features, if the subject is characterized as having 16p13.3 gain; if the subject is characterized as having PTEN deletion; or if the subject is characterized as having 16p13.3 gain and PTEN deletion.

In some embodiments, a higher cancer risk is associated with a reduction in recurrence free survival or metastasis free survival. In some embodiments, the cancer risk is a risk classification of the cancer and the higher cancer risk is associated with the association of the subject with a more aggressive class of the cancer. In one embodiment, methods provided herein allows for further classification into risk sub-groups. In one embodiment, methods provided herein allows for further classification of low to intermediate risk groups into further sub-groups.

The methods described herein can help in characterizing the subject as being associated with a poor prognosis if the subject has the 16p13.3 gain and optionally the PTEN deletion or both chromosomal anomalies. This poor prognostic can be associated with a reduction in survival (including recurrence-free and metastasis-free survival) or an affliction by a more aggressive cancer.

### Prostate Cancer Treatment

The present disclosure also provides treating the subject with adjuvant or neoadjuvant therapy if the subject is characterized as being associated with higher cancer risk. The term “adjuvant therapy” refers to a therapy that is given in addition to the primary or initial therapy to maximize its effectiveness. The term “neoadjuvant therapy” refers to the administration of therapeutic agents before a main treatment.

Examples of primary or main treatment for prostate cancer include but are not limited to surgical therapy, such as, transurethral resection of the prostate (TURP), prostatectomy, transurethral incision of the prostate (TUIP), transurethral vaporization of the prostate (TUVP), photoselective vaporization of the prostate (PVP), prostatic urethral lift (PUL), transurethral microwave therapy (TUMT), water vapor thermal therapy, transurethral needle ablation (TUNA), laser enucleation, prostate artery embolization (PAE), cryosurgery; radiation therapy, such as, external beam radiation therapy, bracytherapy; hormonal therapy, such as, androgen deprivation therapy, antiandrogens, androgen synthesis inhibitors, GnRH antagonists, abiraterone acetate; chemotherapy with cytotoxic agents, or immunotherapy.

In some embodiments, subjects with prostate cancer are treated with radical prostatectomy or radiation therapy as primary therapy.

The term “cytotoxic agent” as used herein refers to a substance that inhibits or prevents the function of cells and/or causes destruction of cells. The term is intended to include radioactive isotopes (e.g. I¹³¹, I¹²⁵, Y⁹⁰, and Re¹⁸⁶), chemotherapeutic agents, and toxins such as enzymatically active toxins of bacterial, fungal, plant or animal origin, or fragments thereof.

A “chemotherapeutic agent” is a chemical compound useful in the treatment of cancer. Examples of chemotherapeutic agents include alkylating agents such as docetaxel, thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard. nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carzinophilin, chromomycins, dactinomycin, daunorubicin, 6-diazo-5-oxo-L-norleucine, doxorubicin, epirubicin, mitomycins, mycophenolic acid, nogalamycin, olivomycins, peplomycin, potfiromycin, puromycin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals 25 such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfomithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2, 2′,2″-trichlorotriethylamine; urethan; vindesine; dacarbazine; mannomustine; mitobronitol; mitolactol; pipobroman; gacytos:ine; arabinoside (“Ara-C”); cyclophosphamide; thiotepa; taxoids, e.g. paclitaxel (TAXOL®, Bristol-Myers Squibb Oncology, Princeton, N.J.) and doxetaxel (Taxotere™, Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; carminomycin; aminopterin; xeloda; ibandronate; CPT-11; topoisomerase inhibitor RFS 2000; difluoromethylomithine (DMFO); retinoic acid; esperamicins; capecitabine; and pharmaceutically acceptable salts, acids or derivatives of any of the above. Also included in this definition are hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone; and anti-androgens such as flutanlide and nilutamide; and pharmaceutically acceptable salts, acids or derivatives of any of the above.

Examples of antiandrogens are flutamide, nilutamide, bicalutamide, enzalutamide, apalutamide, or cyproterone acetate. Examples of androgen synthesis inhibitors are ketoconazole, anioglutethimide or abiraterone. Examples of GnRH antagonists are abarelix or degarelix.

In some embodiments, an adjuvant therapy comprises one or more of surgical therapy, radiation therapy, hormonal therapy, chemotherapy, or immunotherapy. In some embodiments, neoadjuvant comprises one or more of surgical therapy, radiation therapy, hormonal therapy, chemotherapy immunotherapy. In one embodiment, the primary therapy is surgical therapy and/or radiation therapy, and the adjuvant and/or neoadjuvant therapy is one or more of hormonal therapy, chemotherapy, or immunotherapy. In one embodiment, the primary therapy is a first hormonal therapy, chemotherapy, and/or immunotherapy, and the adjuvant and/or neoadjuvant therapy is a second hormonal therapy, chemotherapy, and/or immunotherapy.

In the context of the present disclosure, methods of treating a subject having prostate cancer with an adjuvant or neoadjuvant therapy is provided. The method involves performing the methods described herein on the tumor sample to determine if the subject has a 16p13.3 gain, and characterizing the cancer risk of the subject. If the subject has higher cancer risk, then the subject is administered adjuvant or neoadjuvant therapy. In one embodiment, further microscopy assay is performed to determine if the subject has a PTEN deletion. In some embodiments, the methods include performing a microscopy assay is a fluorescent microscopy assay. In one embodiment, the microscopy assay is FISH.

In some embodiments, characterizing the patient as having higher cancer risk or poor prognosis comprises characterizing the subject as having 16p13.3 gain; characterizing the subject as having PTEN deletion; or characterizing the subject as having 16p13.3 gain and PTEN deletion.

In one embodiment, a subject characterized has having higher cancer risk is treated with abiraterone plus prednisone, enzalutamide or docetaxel. In one embodiment, a subject characterized has having higher cancer risk is treated with ketoconazole plus steroid, mitoxantrone or radionuclide therapy. In one embodiment, a subject characterized has having higher cancer risk is treated with radium²²³. In one embodiment, a subject characterized has having higher cancer risk is treated with docetaxel or mitoxantrone chemotherapy. In one embodiment, a subject characterized has having higher cancer risk is treated with abiraterone plus prednisone, cabazitaxel or enzalutamide. In one embodiment, a subject characterized has having higher cancer risk is treated with docetaxel. In one embodiment, a subject characterized has having higher cancer risk is treated with abiraterone plus prednisone, enzalutamide, ketoconazole plus steroid or radionuclide therapy.

In one embodiment, a subject characterized has having higher cancer risk is treated neoadjuvant therapy or preventative therapy comprising denosumab or zoledronic acid.

### Kits for Assessing Cancer Risk

The present disclosure also provides kits for assessing cancer risk of subject, such as subjects having prostate cancer. The kit includes a first probe capable of specifically recognizing a 16p13.3 chromosome region and a first reference probe capable of specifically recognizing a reference region of chromosome 16. In some embodiments, the first probe hybridizes with a region of chromosome 16 encoding one or more genes encoded in the 16p13.3 chromosome region. In one embodiment, the first probe hybridizes with one or more of PKD1, RAB26, TRAF7, CASKIN1, MLST8, PGP, E4F1, DNASE1L2, ECI1, RNPS1, ABCA3, ABCA17A, CCNF, NTN3, TBC1D24, ATP6V0C, AMDHD2, CEMP1, PDPK1, variants thereof, fragments thereof, or complements thereof. In one embodiment, the first probe is derived from a RP11-20123 BAC clone, variants thereof, fragments thereof, or complements thereof. In some embodiments, the first reference probe hybridizes with a 16qh centromeric region.

In some embodiments, the kit further includes a second probe capable of specifically recognizing a 10q23.3 (PTEN) chromosome region and a second reference probe capable of specifically recognizing a reference region of chromosome 10. In some embodiments, the second probe hybridizes with a region of chromosome 10 encoding the PTEN gene, variants thereof, fragments thereof, or complements thereof. while the second reference probe hybridizes with a 10p11.1-q11.1 centromeric region. In one embodiment, the second probe is derived from a CTD-2557P6 BAC clone, variants thereof, fragments thereof, or complements thereof.

In some embodiments, the probes are fluorescent labeled probes, such as FISH probes. The kit may also include other reagents needed for performing fluorescent microscopy (i.e. FISH), such as further imaging reagents, dyes, contrasting medium, and/or buffers.

The present invention will be more readily understood by referring to the following examples which are given to illustrate the invention rather than to limit its scope.

### Example I—Materials and Methods for 16P13.3 Fish Analysis

Study population and tissue microarray. This study was conducted with the written informed consent of the participants and approval from the Research Ethics Board of McGill University Health Centre (Quebec, Canada, BDM-10-115). This biomarker study was done in accordance with REMARK guidelines (McShane et al., 2005). FFPE RP tissue specimens (n=304) collected between 1993 and 2008 at the McGill University Health Centre were represented on a tissue microarray (TMA) by duplicate 1-mm cores taken from the dominant tumor nodule. The clinical correlates were retrieved from the medical chart and pathologic data were obtained by re-review of all RP cases by a single dedicated genitourinary pathologist. The recent 2014 International Society of Urological Pathology (ISUP) criteria were used for assigning the final grade (Epstein et al, 2016). The clinicopathologic characteristics of the study subjects are summarized in Table 1. Briefly, the mean preoperative serum PSA level for the cohort was 8.60 (±8.21), and the distribution of GS 6, 7, and ≥8 was 20.4%, 70.4%, and 9.2%, respectively. Sixty-four percent of patients belonged to pT2-stage, while 36% were at stage pT3. Cases for which the serum PSA did not fall to undetectable levels postsurgery were considered as surgical failure and were not included in biochemical recurrence analyses (n=14). Patients who received neoadjuvant hormone therapy (n=5) and cases with missing serum PSA data post-surgery were also excluded from BCR analyses (n=15). Biochemical recurrence (BCR) was defined by serum PSA elevation of >0.2 ng/mL after RP (29%), and the recurrence-free interval was defined as the time between the date of surgery and the date of first PSA increase above 0.2 ng/mL. Patients with no BCR were censored at the last follow-up date with a PSA measurement. The median follow-up for the cohort was 118 months (1-253 months, min-max). The metastatic status was confirmed by imaging in patients with clinical signs or symptoms (n=16). The metastasis-free interval was defined as the time between the date of surgery and the date of first metastasis detection. Patients with no signs/symptoms of metastasis were censored at the last follow-up/PSA date. The Cancer of the Prostate Risk Assessment Post-Surgical (CAPRA-S) score was calculated on the basis of the status of six clinicopathologic variables [preoperative PSA, GS, surgical margin (SM), extracapsular extension, seminal vesicle invasion, lymph node invasion], and each patient was assigned to one of the three risk groups: low (0-2), intermediate (3-5), and high (6) according to Cooperberg and colleagues (Cooperberg et al., 2011). Of note, patients who did not undergo a lymph node dissection were deemed to have negative lymph nodes for CAPRA-S score calculation as described previously (Punnen et al., 2014). The prostate cancer DNA CNAs profiling data reported by Taylor and colleagues (Taylor et al., 2010) was used for validation, and the clinical correlates were derived directly from the MSKCC Prostate Cancer Genomics Data Portal.

TABLE 1
Clinicopathologic features of radical prostatectomy
cases represented on the tissue microarray.

| Clinicopathologic variables | Category | n (%) | |
|---|---|---|---|
| Total number of cases | N | 304 | |
| Age (years) | Median | 61 | |
| | Min-max | 43-73 | |
| Preoperative PSA (ng/mL) | n^{a} | 298 | |
| | Mean (±SD) | 8.60 | (±8.21) |
| | PSA < 10 | 232 | (78%) |
| | PSA ≥ 10 | 66 | (22%) |
| GS at surgery | GS 6 | 62 | (20.4%) |
| | GS 7 | 214 | (70.4%) |
| | GS ≥ 8 | 28 | (9.2%) |
| Pathologic stage (T-stage) | pT2 | 195 | (64%) |
| | pT3A | 87 | (29%) |
| | PT3B | 22 | (7%) |
| Surgical margin status | Positive | 91 | (30%) |
| Follow-up (months) | n^{a} | 270 | |
| | Median (min-max) | 118 | (1-253) |
| BCR | n^{a} | 270 | |
| | Positive | 78 | (29%) |
| Metastases | Positive | 16/293^{a} | (5.4%) |

^{a}Values not available for all the 304 cases (n noted for each variable).

Fluorescence in situ hybridization (FISH). Dual-color FISH was performed on TMA sections using as probes, a 16p13.3 specific BAC clone RP11-20123 (BACPAC Resources Center) and the recombinant DNA clone pHuR-195 (ATCC), mapping to the 16qh centromeric region (Moyzis et al., 1987). RP11-20123 and pHuR-195 DNA were labeled with Spectrum OrangedUTP and Spectrum Green-dUTP (Enzo Life Sciences) respectively, using Nick Translation Reaction Kit (Abbott Molecular) and were used to perform FISH on 5-mmTMA sections as described previously (Choucair 2012).

FISH data analysis. To evaluate the 16p13.3 copy number status, fluorescent signals were counted in 100 non-overlapping interphase nuclei for each case (as identified on corresponding H&E) counterstained with ProLong Gold antifade reagent with DAPI (Life Technologies), to delineate nuclei. The 16p13.3 gain was defined as present at a threshold of ≥15% of tumor nuclei containing three or more 16p13.3 locus signals and two pHuR-195 signals, as previously reported (Choucair 2012). Images were acquired with an Olympus IX-81 inverted microscope at X96 magnification, using Image-Pro Plus 7.0 software (Media Cybernetics).

Statistical analysis. Associations between the 16p13.3 gain and clinicopathologic variables were evaluated by Fisher exact test for dichotomous variables and unpaired t test for continuous variables. Kaplan-Meier method and the log-rank test were used to generate and compare recurrence-free survival and metastasis-free survival curves, respectively. Cox regression analyses and the Wald test were used to evaluate univariate and multivariate HRs. The C-index was calculated as described by Harrell and colleagues (Harrell et al., 1996). Analyses were performed using SPSS, WinStat, and R (Version 3.3.2).

### Example II—Results of 16p13.3 Fish Analysis

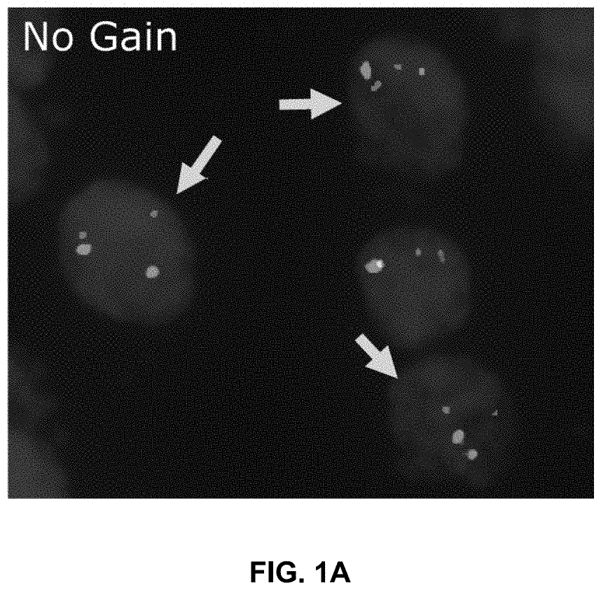

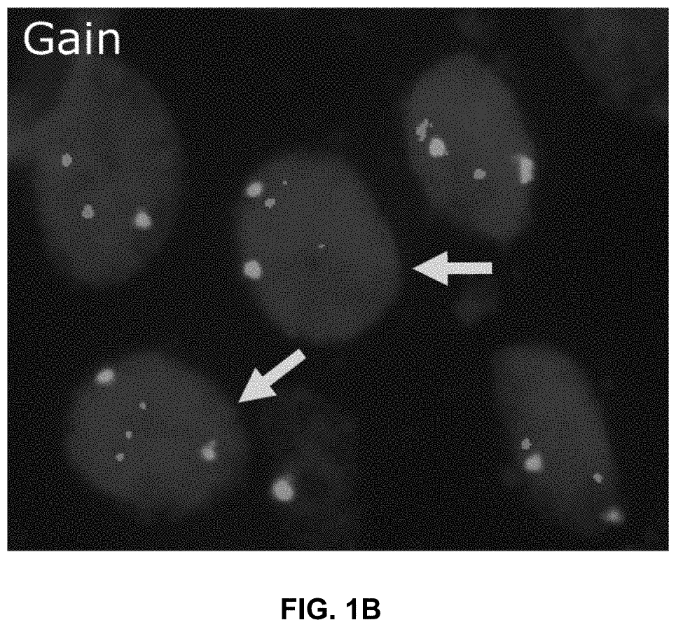

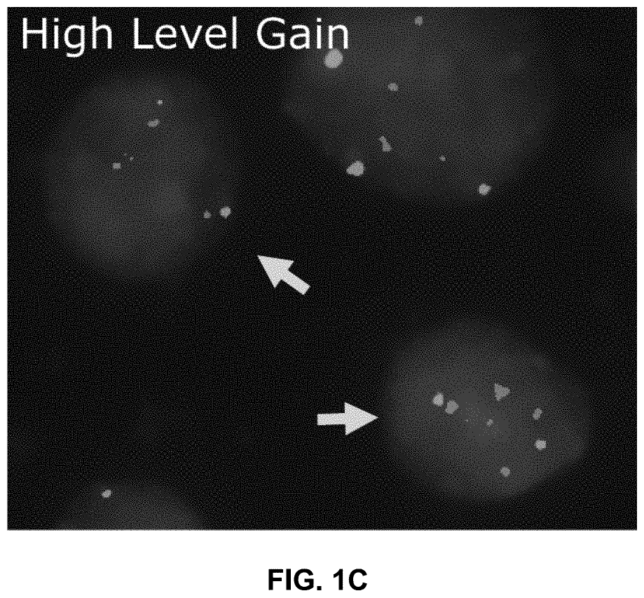

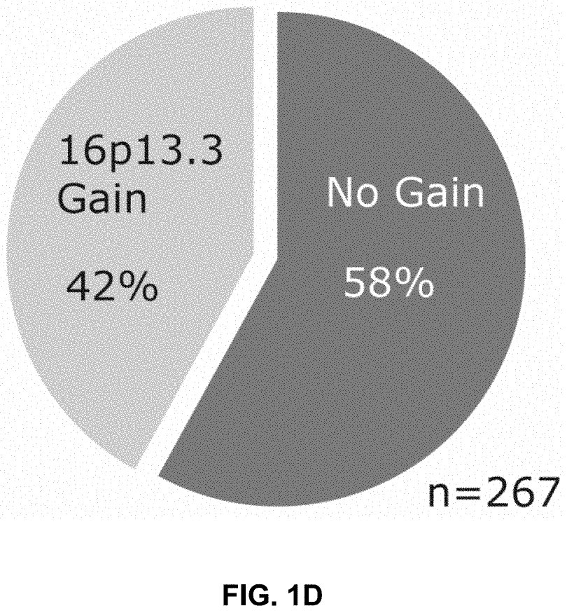

Association of 16p13.3 gain with adverse clinicopathologic features in RP. Dual-color FISH was used to assess CNA at chromosome 16p13.3 on 304 RP specimens represented on a TMA. The clinicopathologic characteristics of the study subjects are summarized in Table 1. A total of 267 primary tumors were scorable by FISH, among which 113 (42%) harbored significant 16p13.3 genomic gain (FIG. 1A to 1D). The 16p13.3 gain was significantly associated with clinicopathologic features of aggressive prostate cancer (Table 2) including high preoperative serum PSA levels (P=0.03), GS (P<0.0001), advanced pT-stage (P<0.0001), and positive SMs (P=0.009). The level of gain was restricted to single copy gain in most specimens, whereas 20 cases with 16p13.3 gain exhibited more than three copies of the locus in at least 10% of their nuclei. As compared with the organ-confined stage-T2 tumors, the advanced stage-T3 tumors harbored significantly higher percentage of nuclei with more than three copies (Mann-Whitney U test, P=0.01).

TABLE 2
Association of 16p13.3 gain with clinicopathologic
features of aggressive prostate cancer.

| | Total Cases | 16p13.3 Status | | | | |
|---|---|---|---|---|---|---|
| Variables_McGill Cohort | n (%) | No gain | | Gain | | P-value |
| 16p13.3 Status | 267 | 154 | (58%) | 113 | (42%) | |
| Age (years) Mean (±SD) | 61.8 (±5.8) | 61.0 | (±6.12) | 60.7 | (±5.64) | 0.68^{†} |
| Preoperative PSA (Mean/±SD) | 261* | 7.6 | (±6.88) | 9.61 | (±8.28) | 0.03^{†} |
| Gleason Score | 267 | | | | | <0.0001^{‡} |
| GS = 6 | 59 | 49 | (83%) | 10 | (7%) | |
| GS = 7 | 185 | 98 | (53%) | 87 | (47%) | |
| GS ≥ 8 | 23 | 7 | (30%) | 16 | (70%) | |
| Pathological T-Stage | 267 | | | | | <0.0001^{‡} |
| pT2 | 176 | 118 | (67%) | 58 | (33%) | |
| pT3A | 72 | 31 | (43%) | 41 | (57%) | |
| pT3B | 19 | 05 | (26%) | 14 | (74%) | |
| Surgical Margins | 267 | | | | | 0.009 |
| Negative | 189 | 119 | (63%) | 70 | (37%) | |
| Positive | 78 | 35 | (45%) | 43 | (55%) | |

^{†}unpaired t-test
^{‡}two-sided Fisher's exact test
*Values not available for all the 267 cases that could be assessed by FISH (n noted for
each variable)

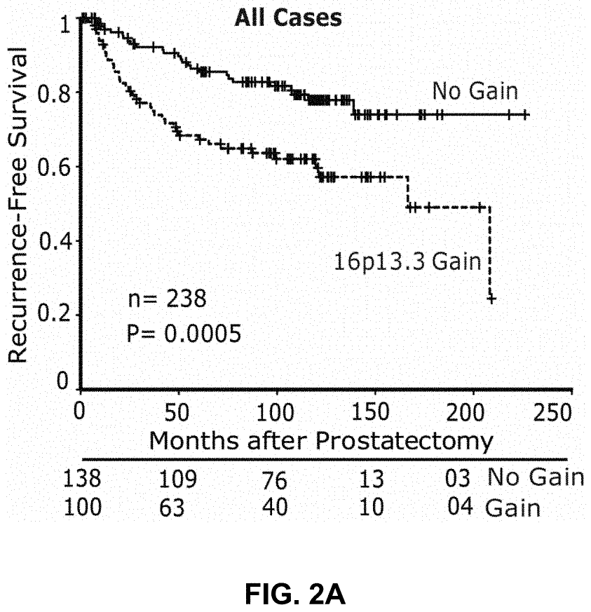

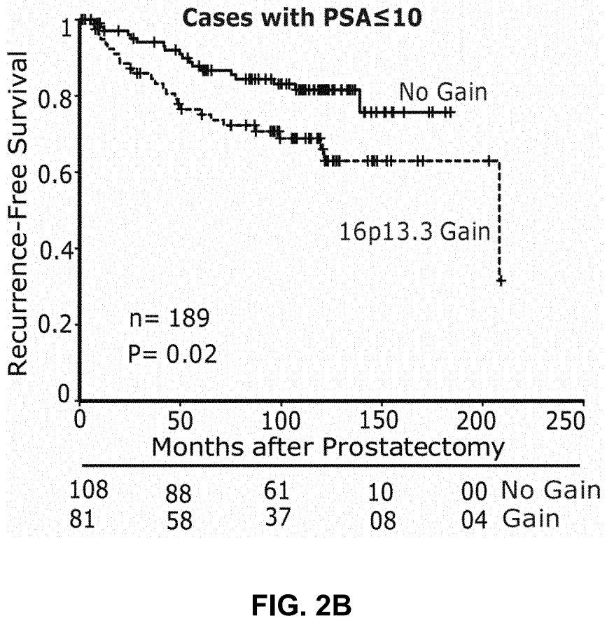

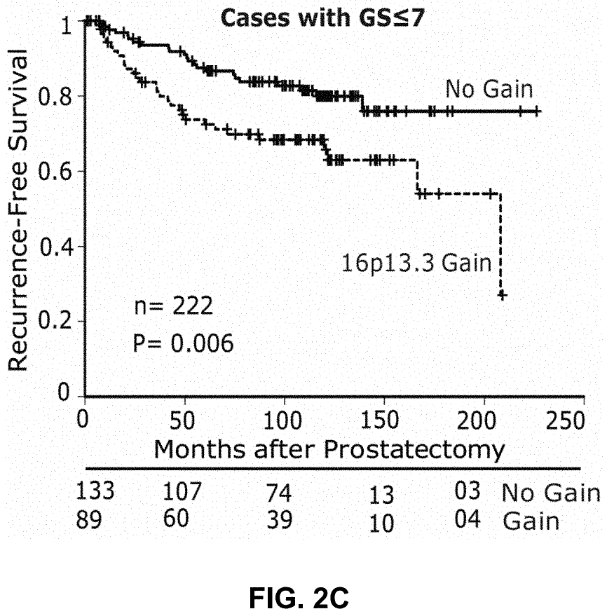

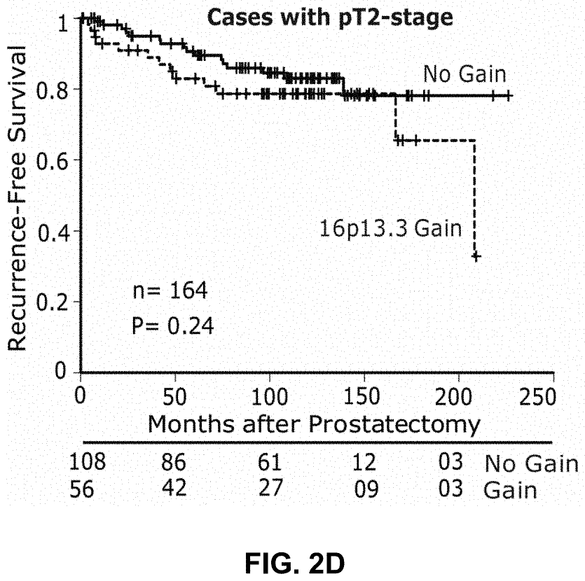

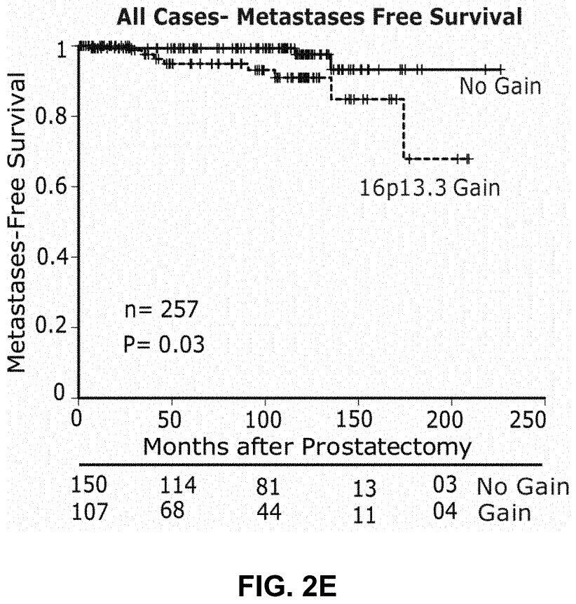

Prognostic significance of 16p13.3 genomic gain in prostate cancer. The impact of the 16p13.3 genomic gain was assessed on clinical outcome using BCR as a surrogate primary endpoint following RP. Of the 238 cases for which both FISH and complete PSA follow-up data were available (median follow up=117 months), 65 (26%) experienced BCR. The Kaplan-Meier analysis revealed that the 16p13.3 gain was significantly associated with shorter BCR-free survival following RP (logrank P=0.0005; FIG. 2A). The 16p13.3 gain was then assessed in terms of its ability to stratify patients with low-intermediate risk, representing a majority of patients encountered in clinical practice. It was observed that 16p13.3 gain status could further risk stratify patients for BCR when considering the subgroup of patients with either preoperative PSA10 or GS7 (log-rank P=0.02 and P=0.006, respectively; FIGS. 2B and 2C), but not significantly in the pT-stage≤T2 subgroup (log-rank P=0.24; FIG. 2D).

Although the main endpoint of this study was BCR, secondary endpoints were also evaluated, such as bone or soft tissue metastases. The 16p13.3 gain status was significantly associated with an increased risk of metastases (log-rank P=0.03; FIG. 2E), supporting its potential utility as a marker of prostate cancer progression.

Improved risk stratification of existing prognostic tools when combined with 16p13.3 gain. The HR for the 16p13.3 gain (HR=2.30; 95% CI, 1.42-3.84; P=0.001) was estimated by univariate Cox regression analysis along with preoperative PSA levels, GS, pT-stage, and SM, which were also significantly associated with BCR (Table 3). In multivariate analysis, the 16p13.3 gain status remained a significant predictor of BCR after adjusting for the above standard clinicopathologic variables (Table 3). These observations were validated in an independent dataset from Taylor and colleagues (Taylor at al., 2010), who reported DNA CNAs by array-CGH analyses of 194 prostate cancer cases with clinical follow-up (Table 4).

TABLE 3
Univariate and multivariate Cox proportional hazards analysis for
16p13.3 gain adjusting for standard clinicopathologic parameters.

| McGill Cohort | Univariate Analysis | | | Multivariate Analysis | | |
|---|---|---|---|---|---|---|
| Variable | HR | 95% CI | P-value* | HR | 95% CI | P-value* |
| Preoperative PSA^{†} | 1.07 | 1.04-1.10 | <0.0001 | 1.05 | 1.02-1.08 | <0.0001 |
| Gleason Score (≥8 | 5.69 | 3.01-10.76 | <0.0001 | 3.06 | 1.47-6.38 | 0.003 |
| vs ≤GS7) | | | | | | |
| pT-stage (T3 vs T2) | 3.46 | 2.10-5.69 | <0.0001 | 1.96 | 1.10-3.48 | 0.02 |
| 16p13.3 Status | 2.30 | 1.40-3.78 | 0.001 | 1.71 | 1.01-2.89 | 0.04 |
| (Gain vs No Gain) | | | | | | |
| Surgical Margin | 2.14 | 1.30-3.51 | 0.003 | 1.53 | 0.88-2.64 | 0.12 |
| (Positive vs Negative) | | | | | | |

^{†}Analyzed as continuous variable;
HR: Hazard ratio;
CI: Confidence Interval
*Wald test

TABLE 4
Univariate and Multivariate Cox Proportional Hazard Analysis
for 16p13.3 Gain in Taylor et al. Validation Dataset

| Taylor et al. Cohort | Univariate Analysis | | | Multivariate Analysis | | |
|---|---|---|---|---|---|---|
| Variable | HR | 95% CI | P-value* | HR | 95% CI | P-value* |
| Preoperative PSA^{†} | 1.005 | 1.002-1.007 | <0.0001 | 1.007 | 1.004-1.01 | <0.0001 |
| Gleason Score (≥8 | 7.60 | 4.42-13.07 | <0.0001 | 6.44 | 3.57-11.61 | <0.0001 |
| vs ≤GS7) | | | | | | |
| pT-stage (T3 vs T2) | 3.42 | 1.95-5.99 | <0.0001 | 2.40 | 1.24-4.64 | 0.009 |
| 16p13.3 Status | 4.95 | 2.09-11.74 | <0.0001 | 4.50 | 1.68-12.04 | 0.003 |
| (Gain vs No Gain) | | | | | | |
| Surgical Margin | 1.82 | 1.07-3.10 | 0.02 | 1.21 | 0.64-2.30 | 0.54 |
| (Positive vs Negative) | | | | | | |

^{†}Analyzed as continuous variable;
HR: Hazard ratio;
CI: Confidence Interval
*Wald test

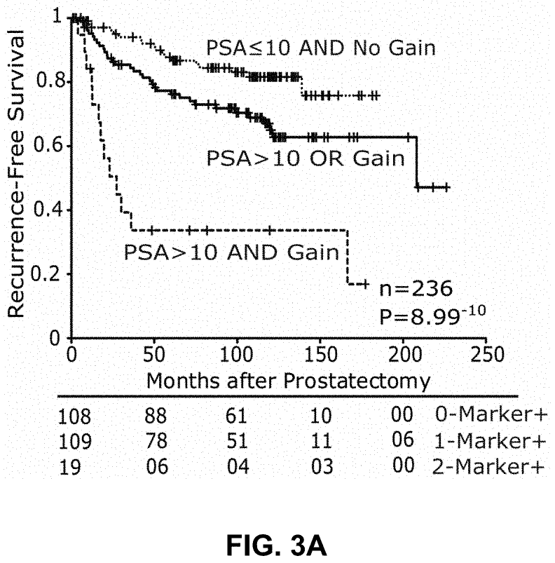

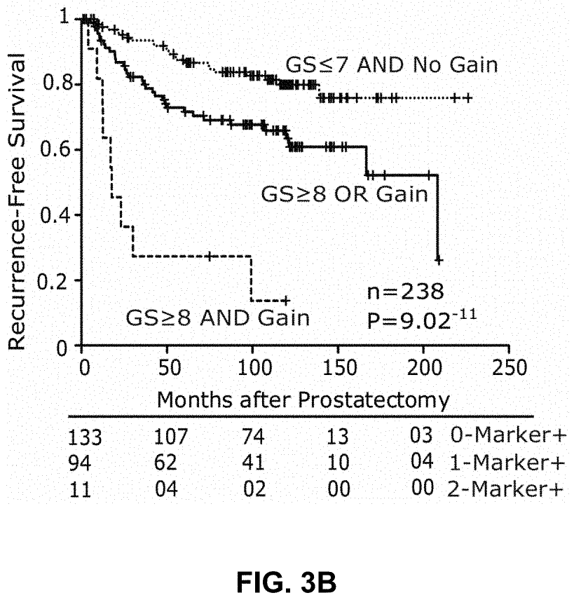

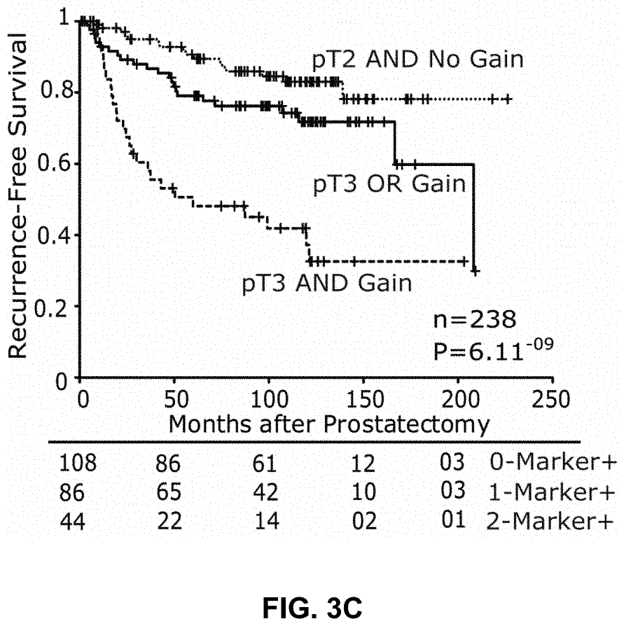

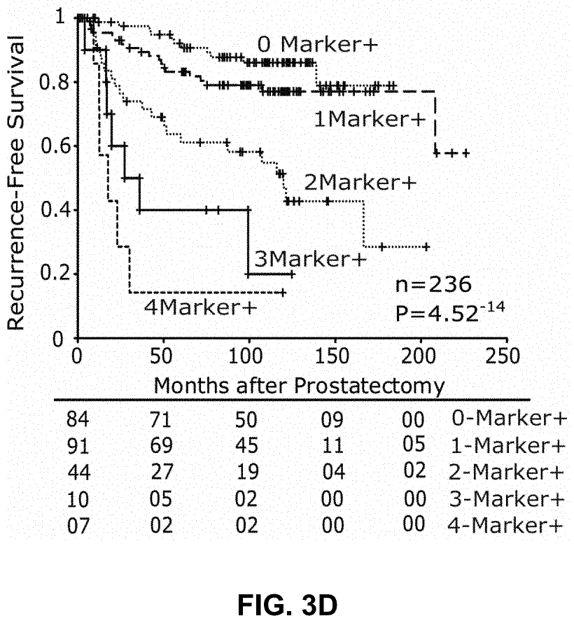

It was further evaluated whether combining 16p13.3 gain status with preoperative PSA, GS, and pT-stage, respectively, would improve patient stratification. The combination of the 16p13.3 gain status with each of these standard clinicopathologic variables segregated prostate cancer cases into three prognostic subgroups (log-rank P<0.0001, respectively; FIG. 3A to 3C) stratified by the number of positive markers: (i) worst prognostic group characterized by presence of both markers; (ii) intermediate prognostic group with either of the two positive markers; and (iii) favorable prognostic group, with both markers absent. Furthermore, the BCR risk stratification significantly improved when all the 4 variables were considered to group the patients (log-rank test, P<0.0001; FIG. 3D).

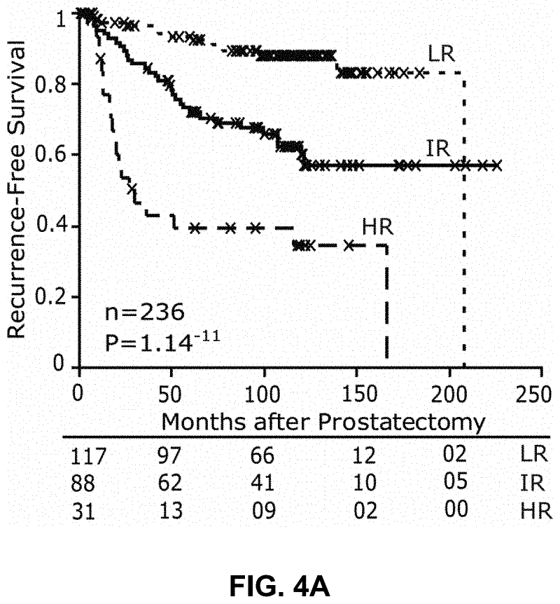

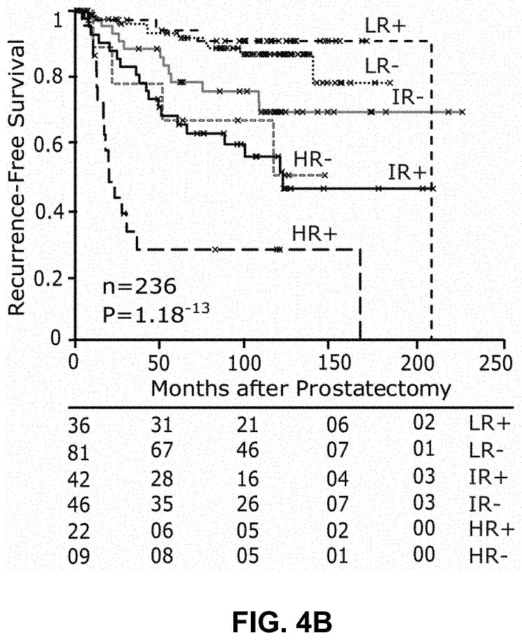

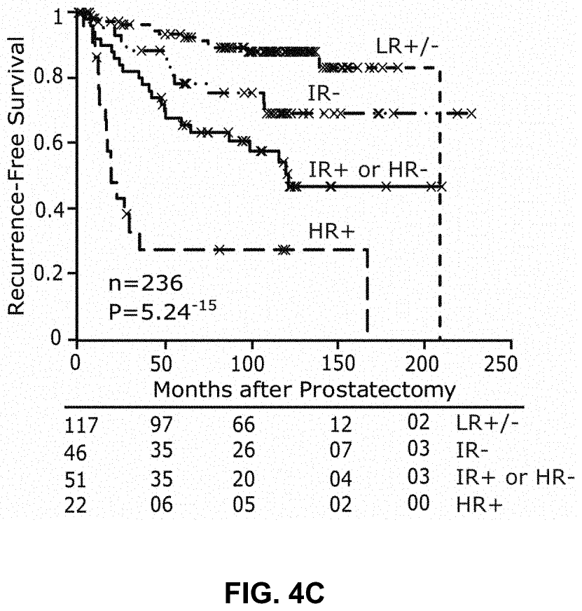

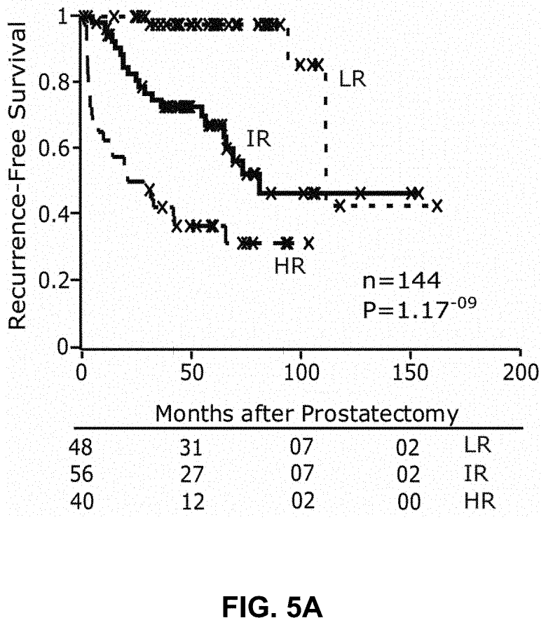

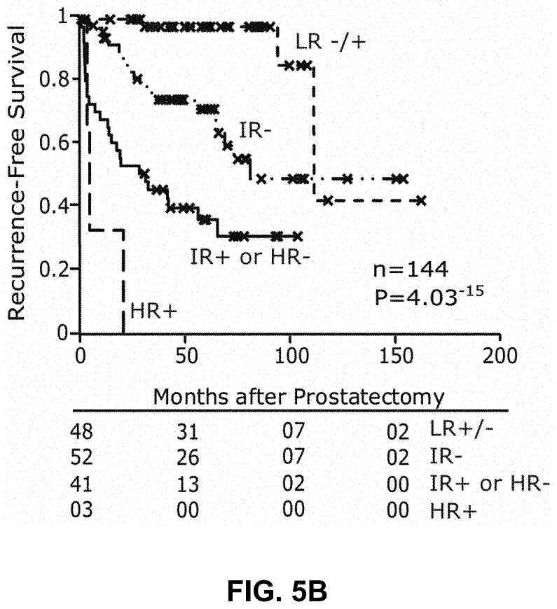

It was then explored whether the 16p13.3 gain status could provide further prognostic information to the CAPRA-S score, a recently developed and validated clinicopathologic tool to predict the risk of recurrence post RP (Cooperberg et al., 2011). In multivariate analysis, the 16p13.3 gain status was a significant predictor of BCR along the CAPRA-S score risk groups (Table 5). As expected, the three risk groups defined by the CAPRA-S score were associated with distinct BCR-free survival probabilities (FIG. 4A). It was then assessed whether the 16p13.3 gain status could further stratify each of these risk groups. Although the 16p13.3 gain status did not further stratify the low-risk group, cases in the intermediate-risk group harboring the gain presented a similar risk of BCR as the high CAPRA-S risk group without this genomic alteration, while those belonging to the high CAPRA-S risk group with the gain had the worst outcome (FIG. 4B). By merging groups with the overlapping risk of BCR, four risk groups were delineated (FIG. 4C). The addition of 16p13.3 gain status to the CAPRA-S score further led to the increase of the C-index as compared with the CAPRA-S score alone (0.78 vs. 0.77). Similarly, in Taylor and colleagues validation dataset, the gain status with CAPRA-S score also identified cases with very high risk of recurrence with a C-index of 0.73 as compared with 0.72 for the CAPRA-S alone (FIGS. 5A and 5B).

TABLE 5
Univariate and multivariate Cox proportional hazards analysis
for CAPRA-S score risk groups and 16p13.3 gain.

| McGill Cohort | Univariate Analysis | | | Multivariate Analysis | | |
|---|---|---|---|---|---|---|
| Variable | HR | 95% CI | P-value* | HR | 95% CI | P-value* |
| CAPRA-S Risk | | | | | | |
| Low (0-2) Ref. | | | <0.0001 | | | <0.0001 |
| Intermediate (3-5) | 3.13 | 1.66-5.91 | 0.0004 | 2.91 | 1.54-5.51 | 0.001 |
| High (≥6) | 8.94 | 4.49-17.79 | <0.0001 | 7.82 | 3.89-15.73 | <0.0001 |
| 16p13.3 Status | | | | | | |
| (Gain vs No Gain) | 2.30 | 1.40-3.78 | 0.001 | 1.81 | 1.09-3.00 | 0.02 |

HR: Hazard ratio;
CI: Confidence Interval
*Wald test

### Example III—Role of 16P13.3 Gain in Prostate Cancer Progression and as Prognostic Biomarker

The detection of 16p13.3 gain in primary prostate cancer specimens as shown above in Examples I and II was in coherence with previously published report, where Choucair and colleagues detected 16p13.3 gain by FISH in 20% of the 46 RP specimens assessed (Choucair et al., 2012). The difference in the proportion of cases harboring the gain between the previous study (20%) and the present study (42%) might be attributed to the small sample size of the earlier study (n=46) or reflected real biological differences between these two independent patient sets. The majority of cases with 16p13.3 gain harbored a single extra copy. Single copy gains of loci or entire chromosome are known recurrent events in other types of cancer that effectively contribute to tumor phenotypes (Abel et al., 1999; An et al., 2014; Bown et al., 1954; Towle et al., 2014). A few cases harbored more than a single copy gain, and they were more prevalent in the pT3-stage tumors.

The 16p13.3 genomic gain was associated with clinicopathologic features of aggressive prostate cancer, such as high GS and preoperative PSA levels. Patients harboring 16p13.3 gain were more than twice as likely to experience BCR following RP than those without the gain. Combining the 16p13.3 gain status with individual clinicopathologic markers significantly improved BCR risk stratification in this study, wherein the incremental number of positive variables was associated with a higher risk of BCR, which reached its maximum for patients with four adverse factors, including the 16p13.3 gain. This improved stratification was observed in patients with intermediate and high risk of disease progression based on their CAPRA-S score. These results are in line with previous reports showing that genomic markers can further stratify subsets of patients classified by the CAPRA-S score (Cooperberg et al., 2015; Lennartz et al., 2016). The fact that the CAPRA-S score is already a very strong multiparameter predictor of BCR and that the low-risk group was not further stratified by the 16p13.3 gain may explain the limited increase of the C-index observed by the addition of this single variable to the model. Further validation studies are warranted on larger cohorts to further evaluate the added prognostic value of the 16p13.3 gain to the clinicopathologic predictors. Nevertheless, the addition of the 16p13.3 gain status to CAPRA-S identified a subset of patients at very high risk of recurrence, who may benefit from adjuvant treatments after RP.

Although very few metastatic events were observed in the McGill cohort (approximately 5%), the 16p13.3 gain was also predictive of the increased risk of developing distant metastases following RP. Recently, using the whole-genome sequencing approach, Beltran and colleagues detected the 16p13.3 gain in 52% of the metastatic CRPC tumor samples (Beltran et al., 2016). These results are in line with previous studies detecting gain in about 50% prostate cancer lymph node metastases, an overrepresentation as compared with unpaired primary tumors (Lapointe et al., 2007; Choucair et al., 2012). These observations further support a role of 16p13.3 gain in cancer progression and warrant future studies in the context advanced diseases and response to therapies including androgen ablation.

The association of 16p13.3 gain with features of aggressive tumors was in line with studies in the breast (Maurer et al., 2009), lung (Shen et al., 2008), and colon cancer (Mampaey et al., 2015), wherein the 16p13 gain was linked to poor prognosis. The minimal region of 16p13.3 gain (described in Choucair et al. 2012) spans 19 genes. PDPK1, encoding PDK1, is a likely driver of the gain but other genes involved in other types of cancer reside at this locus as well (Choucair et al., 2012). Of these, RAB26 is a Ras oncogene family member found to be upregulated in non-small cell lung carcinoma (Valk et al., 2010) and uveal melanoma (Marshall et al., 2007). Similarly, CCNF (G2-mitosis-specific cyclin-F) was reported to be overexpressed in breast and esophageal cancer, respectively (Tamoto et al., 2004; Williams et al., 2008). ABCA3, a known drug efflux pump belonging to the p-gp family, is overexpressed in acute myeloid leukemia (AML) and different cancer cell lines. Notably, ABCA3 overexpression conferred drug resistance in these AML cases (Chapuy et al., 2008; Steinbach et al., 2006; Yasui et al., 2004). The relevance of these genes in prostate cancer remains to be investigated through functional studies.

One application for the 16p13.3 gain is a FISH biomarker to identify patients requiring adjuvant or neoadjuvant therapies and to improve pretreatment prognostication given that accurate GS can be challenging to obtain on biopsies. Studies on needle biopsy cohort are desirable for its implementation in the presurgical setting. The spatial resolution afforded by a histology-based assay such as FISH would facilitate sensitive assessment of individual cancer foci (Bishop et al., 2010; Gozzetti et al., 2000) in a context of tumor heterogeneity and bypass the need for nucleic acid extraction from bulk tumor tissue required by several recently developed commercial assays based on gene expression signatures, such as Prolaris and Decipher (Cuzick et al., 2012; Irshad et al., 2013; Klein et al, 2014; Klein et al., 2016).

Taken together, the results support a role for 16p13.3 gain in prostate cancer progression and as a relevant prognostic biomarker. Incorporating 16p13.3 gain status with routinely used clinicopathologic variables allows for improvements to stratifying patients into different prognostic groups.

### Example IV—Materials and Methods for PTEN Fish Analysis

Study population and tissue microarray. This study was done in compliance with the REMARK guidelines (McShane et al., 2005) and approved by the Research Ethics Board of the McGill University Health Centre (BDM-10-115) with the written informed consent of the participants. A set of 332 de-identified formalin-fixed paraffin-embedded (FFPE) radical prostatectomy specimens collected between 1993 and 2008 at the McGill University Health Centre were represented on a tissue microarray by duplicate 1 mm cores extracted from the dominant tumor nodule. Dominant nodule was defined as generally the largest nodule. In cases in which a smaller nodule was considered to be prognostically more significant (higher grade or stage), this smaller nodule was considered to be dominant.

TABLE 6
Clinicopathologic features of radical prostatectomy
cases represented on the tissue microarray.

| Clinicopathologic | | | |
|---|---|---|---|
| variables | Category | n (%) | |
| Total number of cases | n | 332 | |
| Age (years) | Median | 61 | |
| | Min-max | 43-73 | |
| Preoperative serum | n^{a} | 327 | |
| prostate-specific antigen | Mean (±SD) | 8.66 | (±8.27) |
| (ng/ml) | PSA ≤ 10 | 253 | (77%) |
| | PSA > 10 | 74 | (23%) |
| Gleason grade groups at | Group 1 (3 + 3) | 70 | (21%) |
| surgery | Group 2 (3 + 4) | 153 | (46%) |
| | Group 3 (4 + 3) | 78 | (24%) |
| | Group 4 (8) | 11 | (3%) |
| | Group 5 (≥9) | 20 | (6%) |
| Pathological stage | pT2 | 219 | (66%) |
| (T-stage) | pT3a | 91 | (27%) |
| | pT3b | 22 | (7%) |
| Surgical margin status | Positive | 97 | (29%) |
| Follow-up (months) | n^{a} | 297 | |
| | Median (min-max) | 116 | (1-253) |
| Biochemical recurrence | n^{a} | 297 | |
| | Positive | 81 | (27%) |
| Distant matastases | Positive | 16/321^{a} | (5%) |

^{a}Values not available for all the 332 cases (n noted for each variable)

The clinical information was retrieved from the medical charts and the pathological correlates were obtained after re-review of all the radical prostatectomy cases by a single dedicated genitourinary pathologist. The final Gleason grade was assigned according to the latest International Society of Urological Pathology/World Health Organization recommendations (Humphrey et al., 2016). The clinicopathologic characteristics of 303 of the 332 cases (see Example I) and those of the entire expanded cohort are summarized in Table 6. The mean preoperative serum prostate-specific antigen level was 8.66 (±8.27) and the distribution of Gleason grade group 1 (Gleason score 6), 2 (Gleason score 3+4), 3 (Gleason score 4+3), 4 (Gleason score 8), and 5 (Gleason score 9) was 21%, 46%, 24%, 3%, and 6%, respectively. Sixty-six percent of patients were at stage pT2 while 34% belonged to stage pT3. Patients receiving neoadjuvant hormone therapy (n=6) and cases with missing serum prostate-specific antigen data post-radical prostatectomy (n=15) were not included in the biochemical recurrence analyses. Surgical failure cases (n=14), for which the serum prostate-specific antigen did not fall to undetectable levels post-radical prostatectomy, were also excluded from the biochemical recurrence analyses. No patient had received adjuvant radiation therapy after surgery. The primary endpoint of the study was biochemical recurrence and was defined by a serum prostate-specific antigen elevation of >0.2 ng/ml following radical prostatectomy (27%). The recurrence-free interval was defined as the time between the surgery date and the date of the first prostate-specific antigen increase above 0.2 ng/ml. Patients without biochemical recurrence event were censored at the last follow-up date with prostate-specific antigen measurement. The median follow-up for the cohort was 116 months (1-253 months, min-max). Metastasis status was evaluated and confirmed by imaging in patients with clinical symptoms (n=16). The metastasis-free interval was defined as the period between the surgery date and the date of first metastasis detection and patients without signs/symptoms related to metastasis were censored at the last follow-up/prostate-specific antigen date. The CAPRA-S(Cancer of the Prostate Risk Assessment Post-Surgical) score was calculated from the status of six clinicopathologic variables [preoperative prostate-specific antigen, Gleason score, surgical margins, extracapsular extension, seminal vesicle invasion, lymph node invasion], and each patient was assigned to one of the three risk groups: low (0-2), intermediate (3-5), and high (6) according to Cooperberg et al. (2011). Of note, patients who did not undergo a lymph node dissection were considered to have negative lymph node for CAPRA-S score calculation as previously described (Punnen et al., 2014). The chromosome 16p13.3 gain data of Examples I to III was used for the combinatorial approach.

Fluorescence in situ hybridization (FISH). The BAC clone CTD-2557P6 (BACPAC Resources Center, Oakland, Calif.) mapping to the PTEN gene on the chromosome 10q23.3 region and commercially available CEP10 Spectrum Green probe (CEP 10, Abbott Molecular, Abbott Park, Ill.), which spans the 10p11.1-q11.1 centromeric region were used to perform dual-color FISH on the 5 μm tissue microarray sections as described previously (Choucair, Ejdelman, et al., 2012). The CTD-2557P6 DNA was labeled with the Spectrum Orange-dUTP (Enzo Life Science, Farmingdale, N.Y.) using the Nick Translation Reagent Kit (Abbott Molecular) as per the kit manual.

FISH data analysis. To evaluate the PTEN copy number status, fluorescent signals were counted in 100 non-overlapping interphase nuclei for each case (as identified on corresponding H&E) counterstained with ProLong® Gold antifade reagent with DAPI (Life Technology, CA), to delineate nuclei. The PTEN deletion was defined as ≥15% of tumor nuclei containing one or no PTEN locus signal and by the presence of two CEP10 signals as previously reported (Choucair, Ejdelman, et al., 2012). A tumor was considered homozygous-deleted if 15% of tumor nuclei had no PTEN locus signals and two CEP10 signals. Images were acquired with an Olympus IX-81 inverted microscope at ×96 magnification, using Image-Pro Plus 7.0 software (Media Cybernetics, Rockville, Md.).

Statistical analysis. The association between copy number alterations and the clinicopathologic indicators were assessed by Fisher's exact test for categorical variables and unpaired t-test for continuous variables. Kaplan-Meier curves were generated for biochemical recurrence-free and metastasis-free survival analysis. The log-rank test was used to evaluate the significance of differences between the stratified survival functions. Cox regression analyses were used to evaluate univariate hazard ratios and multivariate Cox proportional hazards regression analysis was performed to identify independent predictors of biochemical recurrence. The C-index was calculated as described by Harrell et al. (1996). Analyses were performed using SPSS, WinStet, and R (Version 3.3.2).

### Example V—Results of PTEN Fish Analysis

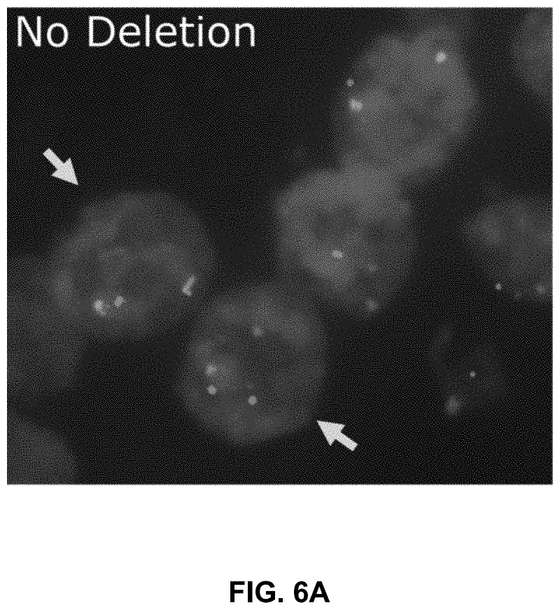

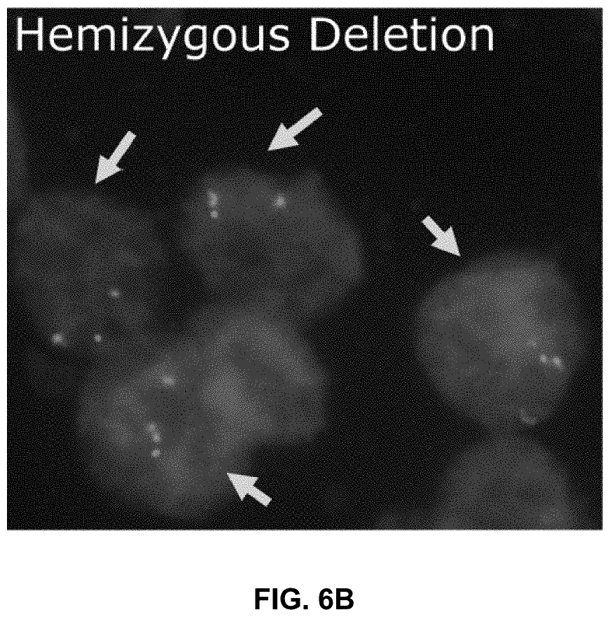

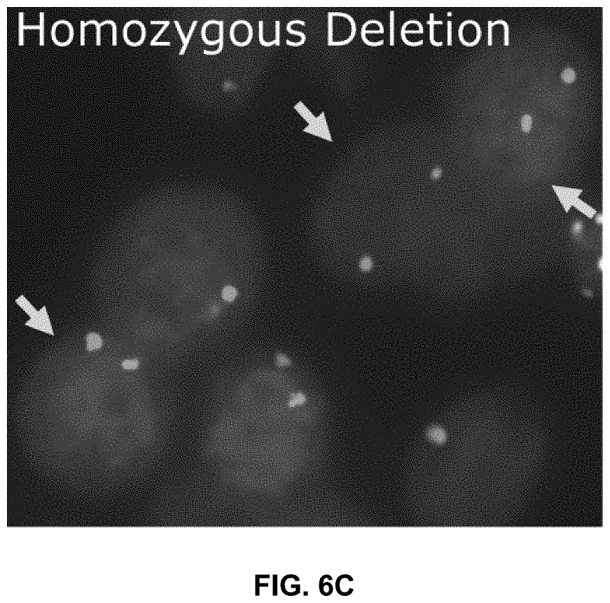

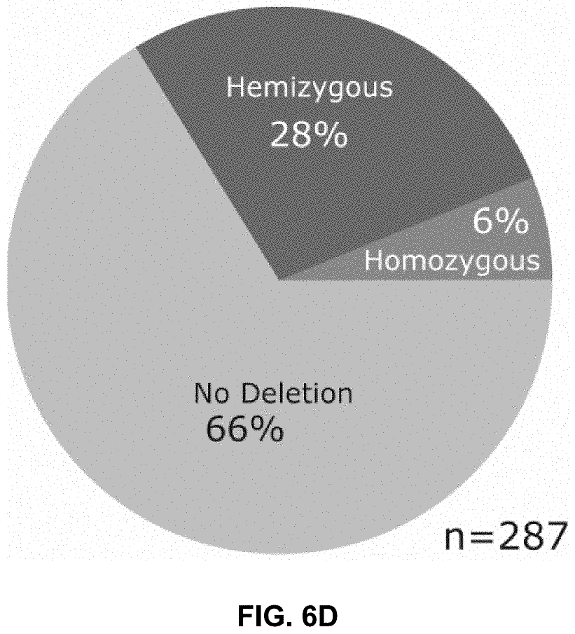

Association of PTEN deletion status with adverse clinical outcome post-radical prostatectomy. The 10q23.3 (PTEN) deletion status was assessed using dual-color FISH on 332 radical prostatectomy specimens represented on a tissue microarray. The clinicopathologic features of these patients are summarized in Table 6. The PTEN deletion status could be successfully assessed in 287 tumors arrayed out, of which 97 (34%) harbored a PTEN genomic deletion. The PTEN deletion status was consistent across duplicate TMA cores evaluated. Of the cases with PTEN deletion, 80 (28%) were hemizygous deleted while 17 (6%) harbored a homozygous PTEN deletion (FIG. 6A to 6D). Of note, 15 out the 17 cases that was identified as homozygous deleted for PTEN also harbored a significant number of nuclei (15%) showing a hemizygous deletion within the tissue microarray core. Preliminary analysis indicated that these cases with homozygous deletion were not different than cases harboring only a hemizygous deletion in term of their association with adverse pathology and poor outcome (not shown). Therefore hemizygous and homozygous PTEN deletion cases were considered together as a single group for the analyses presented in this report. As shown in Table 7, the PTEN deletion status was significantly associated with high Gleason grade group (P=0.0001) and advanced pT-stage (P=0.001).

TABLE 7
Association of PTEN deletion status with clinicopathologic
features of aggressive prostate cancer.

| | Total | | | | | |
|---|---|---|---|---|---|---|
| Clinicopathologic | cases | PTEN status | | | | |
| variables | n (%) | No deletion | | Deletion | | P-value |
| PTEN status | 287 | 190 | (66%) | 97 | (34%) | |
| Gleason grade groups | 287 | | | | | 0.0001 |
| Group 1 (GS 3 + 3) | 61 | 52 | (85%) | 9 | (15%) | |
| Group 2 (GS 3 + 4) | 132 | 90 | (68%) | 42 | (32%) | |
| Group 3 (GS 4 + 3) | 67 | 37 | (55%) | 30 | (45%) | |
| Group 4 and 5 | 27 | 11 | (41%) | 16 | (59%) | |
| (GS ≥ 8) | | | | | | |
| Pathological T-stage | 287 | | | | | 0.001 |
| pT2 | 189 | 138 | (73%) | 51 | (27%) | |
| pT3a | 77 | 43 | (56%) | 34 | (44%) | |
| pT3b | 21 | 9 | (43%) | 12 | (57%) | |
| Preoperative prostate- | 282 | 7.95 | (±7.60) | 9.03 | (±7.24) | 0.25^{a} |
| specific antigen | | | | | | |
| (mean/±standard | | | | | | |
| deviation) | | | | | | |
| Surgical margin status | 287 | | | | | 0.30 |
| Negative | 200 | 136 | (68%) | 64 | (32%) | |
| Positive | 87 | 54 | (62%) | 33 | (38%) | |

P-value calculated by Fisher exact test
Number of cases that could be assessed (n) noted for each variable
^{a}Unpaired t-test

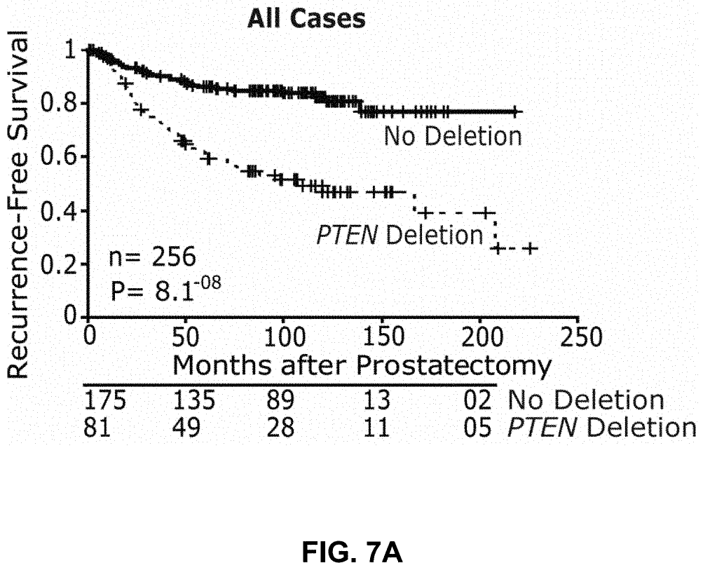

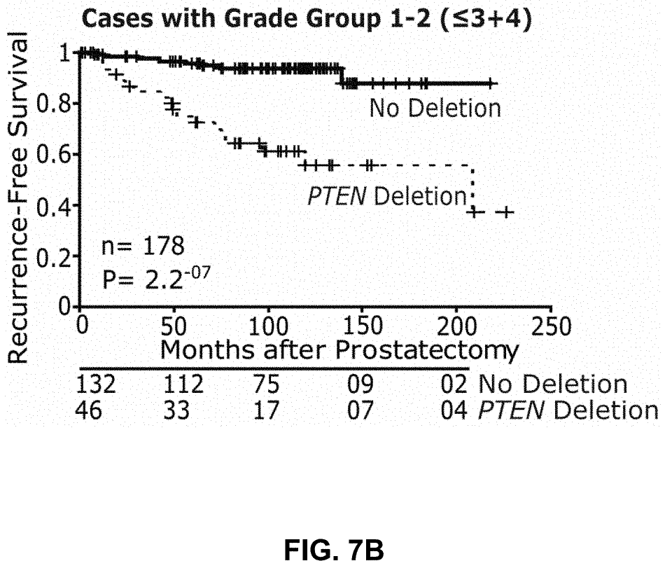

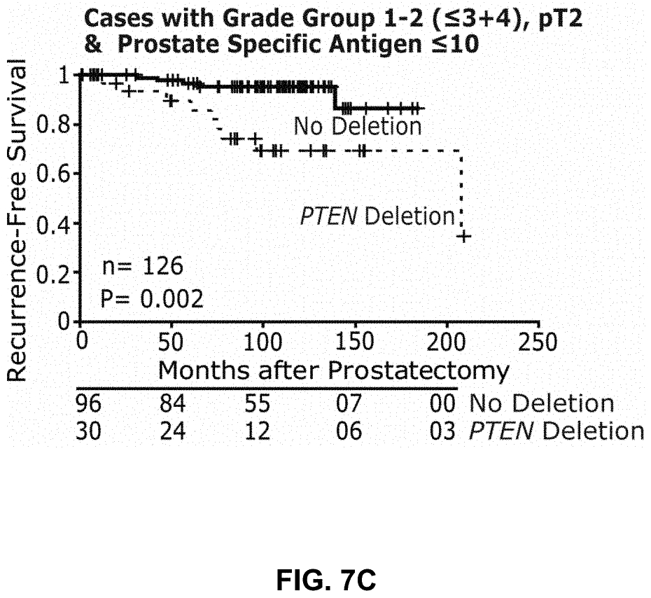

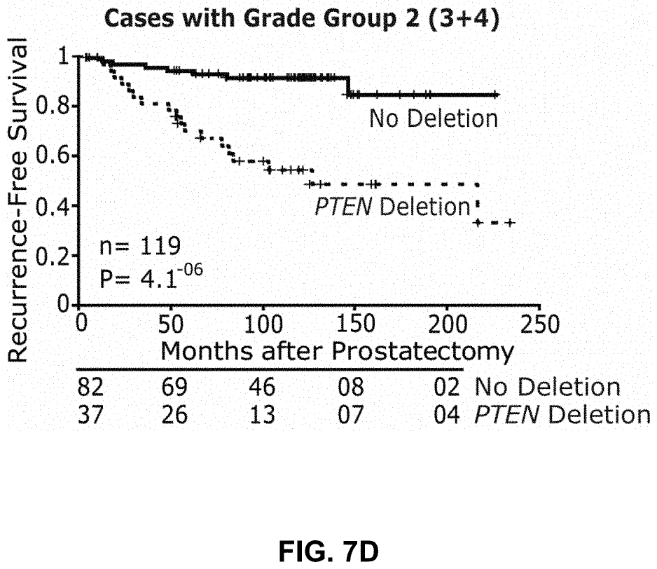

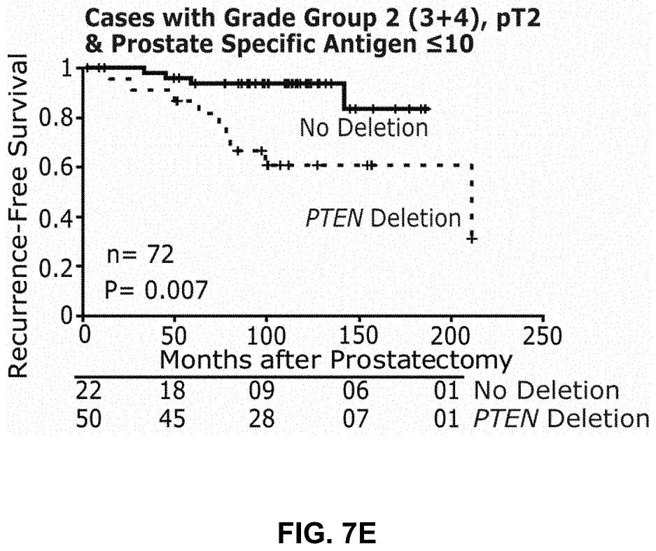

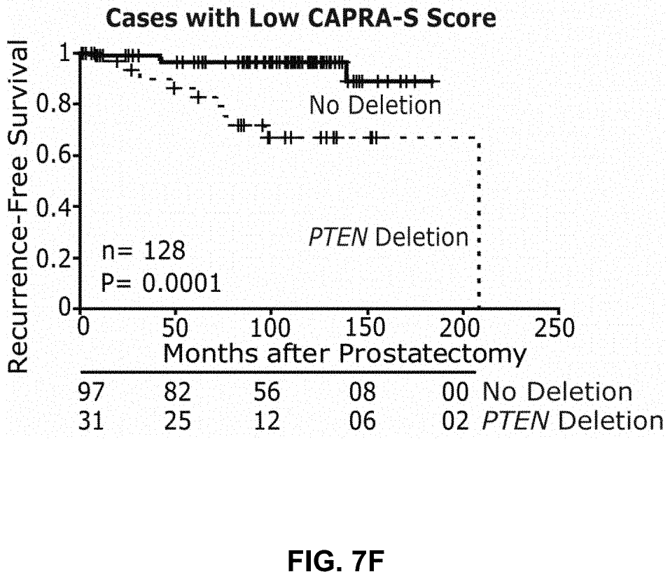

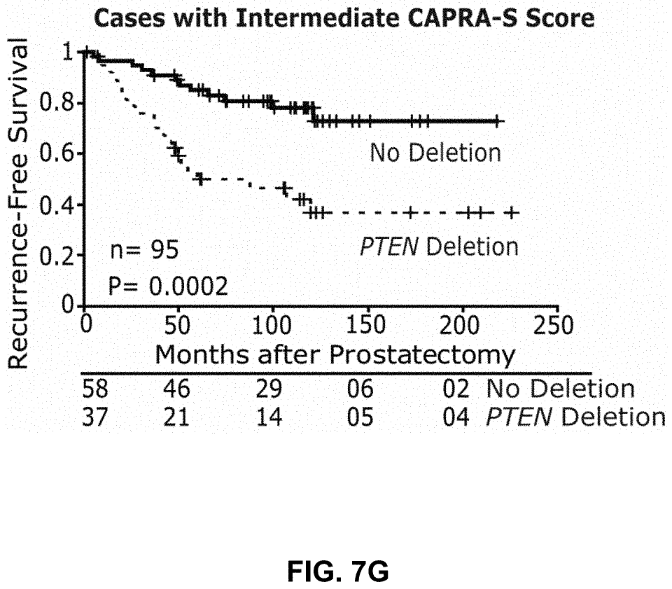

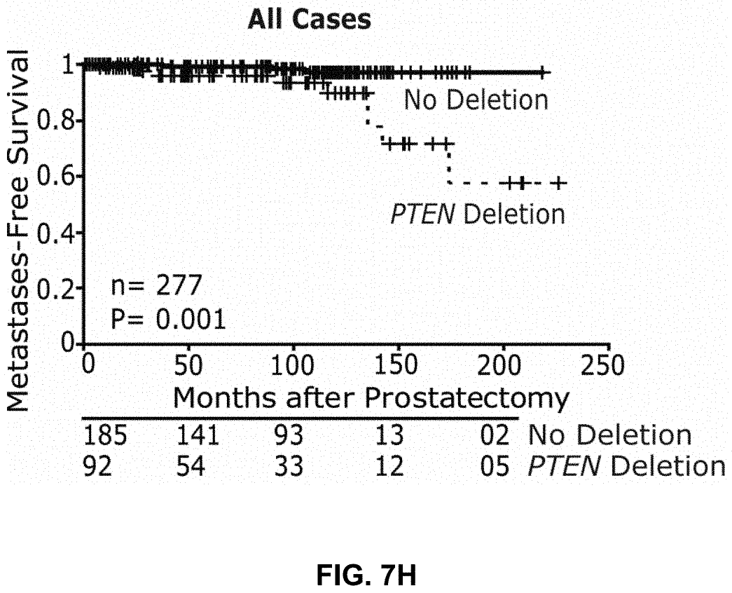

The prognostic significance of PTEN genomic status was first evaluated using biochemical recurrence as a surrogate primary endpoint post-radical prostatectomy. PTEN FISH status and complete prostate-specific antigen follow-up data were available for 256 radical prostatectomy cases, out of which 69 (27%) experienced biochemical recurrence. The PTEN genomic deletion status emerged as a significant predictor of early biochemical recurrence following radical prostatectomy (log-rank P<0.0001; FIG. 7A) independent of the standard clinicopathologic prognostic indicators like Gleason grade group, pT-stage, preoperative prostate-specific antigen level, and surgical margin status in a multivariate Cox analysis (hazard ratio: 3.00, 95% confidence interval: 1.81-4.99; P<0.0001; Table 8, (A)).

Clinical significance of PTEN genomic deletion in low-intermediate risk prostate cancer patients. The ability of the PTEN deletion status was assessed to predict biochemical recurrence (BCR) risk in a clinically relevant subset of patients belonging to grade groups 1 and 2 (≤3+4) according to the latest International Society of Urological Pathology/World Health Organization Gleason grading recommendations (Humphrey et al., 2016). The PTEN deletion status was significantly associated with biochemical recurrence in patients of grade group 1-2 (3+4, log-rank, P<0.0001, FIG. 7B) including those that were also of stage pT2 and with preoperative prostate-specific antigen 10 (log-rank, P=0.002, FIG. 7C). Furthermore, the PTEN deletion was significantly linked to biochemical recurrence in a subgroup of grade group 2 (3+4, log-rank, P<0.0001, FIG. 7D) even with favorable stage pT2 and prostate-specific antigen 10 (log-rank, P=0.007, FIG. 7E). There was an insufficient number of biochemical recurrence events (n=1) in grade group 1 (Gleason score 6) to allow subgroup analysis and the PTEN deletion status did not further stratify grade group 3-5 (≥4+3, not shown). It was then assessed if the PTEN genomic deletion status could further stratify the risk groups defined by the clinically validated clinicopathologic CAPRA-S score to predict biochemical recurrence postradical prostatectomy (Cooperberg et al., 2011). The multivariate analysis showed that the PTEN deletion was a significant predictor of biochemical recurrence along CAPRA-S score risk groups (hazard ratio: 2.84, 95% confidence interval: 1.75-4.63; P<0.0001, Table 8, (B)). PTEN deletion identified a subset of patients with a greater risk of biochemical recurrence among those of low and intermediate CAPRA-S score risk groups (FIGS. 7F and 7G), log-rank, P=0.0001 and P=0.0002, respectively). The association of PTEN deletion with bone or soft tissue metastases (an important adverse secondary endpoint) was further evaluated. PTEN deletion status was indeed significantly associated with an increased risk of distant metastasis (log-rank P=0.001, FIG. 7H), further supporting its potential clinical utility as a marker of prostate cancer progression.

TABLE 8
Univariate and multivariate Cox proportional hazard analysis predicting
biochemical recurrence for PTEN deletion status adjusted for (A)
standard clinicopathologic parameters and (B) CAPRA-S score.

| | Univariate analysis | | Multivariate analysis | |
|---|---|---|---|---|
| | Hazard ratio (95% | | Hazard ratio (95% | |
| Variables | confidence interval) | P-value | confidence interval) | P-value |

(A) Standard clinicopathologic parameters

| | | | | | | |
|---|---|---|---|---|---|---|
| PTEN status (deleted vs. | 3.47 | (2.14-5.63) | <0.0001 | 3.00 | (1.81-4.99) | <0.0001 |
| non-deleted) | | | | | | |
| Preoperative prostate-specific | 1.06 | (1.04-1.09) | <0.0001 | 1.05 | (1.01-1.08) | 0.005 |
| antigen^{a} | | | | | | |

Gleason grade

| | | | | | | |
|---|---|---|---|---|---|---|
| Group 3-5 (≥4 + 3) vs. | 4.75 | (2.91-7.78) | <0.0001 | 2.60 | (1.44-4.67) | 0.001 |
| group 1-2 (≤3 + 4) | | | | | | |
| pT-stage (T3 vs. T2) | 3.32 | (2.05-5.38) | <0.0001 | 1.53 | (0.87-2.67) | 0.137 |
| Surgical margin (positive vs. | 2.30 | (1.43-3.72) | 0.001 | 1.88 | (1.12-3.13) | 0.016 |
| negative) | | | | | | |
| Age at surgery^{a} | 1.02 | (0.98-1.07) | 0.256 | 0.98 | (0.94-1.03) | 0.494 |

(B) CAPRA-S score

| | | | | | | |
|---|---|---|---|---|---|---|
| PTEN status (deleted vs. | 3.47 | (2.14-5.63) | <0.0001 | 2.84 | (1.75-4.63) | <0.0001 |
| non-deleted) | | | | | | |

CAPRA-S risk

| Low (0-2) reference | — | | <0.0001 | — | | <0.0001 |
|---|---|---|---|---|---|---|
| Intermediate (3-5) | 3.40 | (1.81-6.36) | <0.0001 | 3.00 | (1.60-5.64) | 0.001 |
| High (≥6) | 10.65 | (5.42-20.91) | <0.0001 | 8.95 | (4.53-17.67) | <0.0001 |

^{a}Analyzed as a continuous variable;
P-value: Wald test

### Example VI—16P13.3 Gain and Pten Deletion Combination as Prognostic Biomarkers

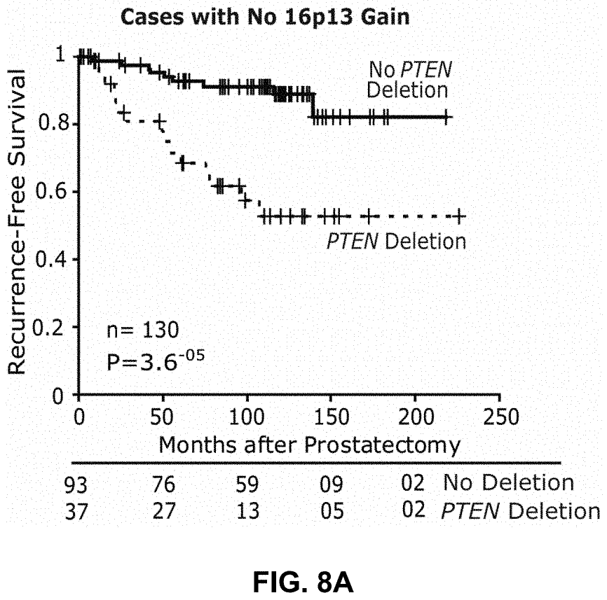

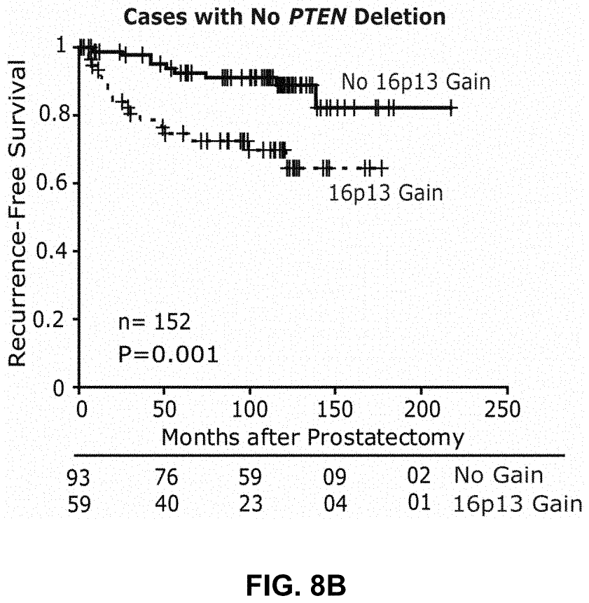

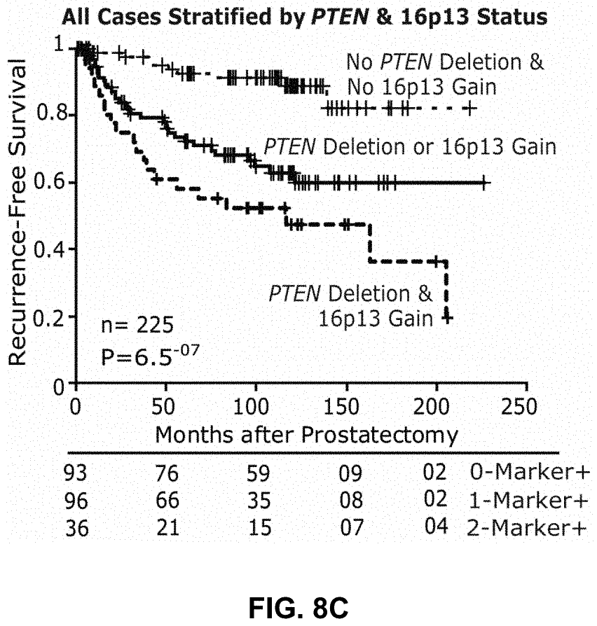

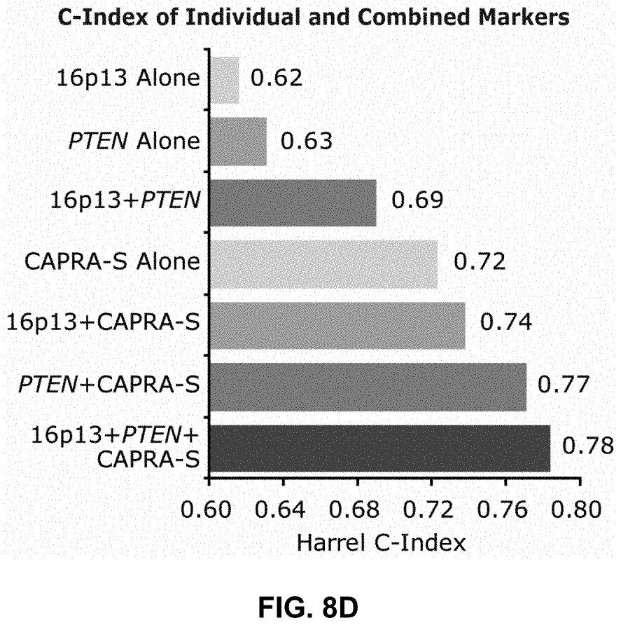

Improved biochemical recurrence risk stratification upon combining 16p13.3 gain with PTEN deletion. As discussed above, 16p13.3 genomic gain status is associated with aggressive clinicopathologic features of prostate cancer (Choucair et al., 2012), as well as with poor clinical outcome. A set of 251 cases for which both 16p13.3 gain and PTEN deletion data were available was used for the combinatorial PTEN-16p13.3 co-alteration analyses. It was first tested whether PTEN deletion status could further stratify patients without 16p13.3 gain. As shown in FIG. 8A, cases with PTEN deletion have an increased risk of biochemical recurrence among this subgroup (log-rank, P<0.0001). Interestingly, amongst patients with no PTEN deletion, the 16p13.3 gain further identified a subset of patients at high risk of recurrence (FIG. 8B, log-rank, P=0.001). Cases were then grouped based on their PTEN-16p13.3 co-alteration status-(0) no PTEN deletion and no 16p13.3 gain, (1) PTEN deletion or 16p13.3 gain, and (2) PTEN deletion and 16p13.3 gain. Kaplan-Meier analysis demonstrated that the PTEN-16p13.3 co-alteration status further segregated prostate cancer cases in three distinct prognostic subgroups (logrank P<0.0001, FIG. 8C) stratified by the number of positive markers: the favorable prognostic group with no alterations in PTEN and 16p13.3, the intermediate prognostic group with one alteration in either PTEN or 16p13.3, and the worst prognostic group with two alterations (PTEN and 16p13.3). Moreover, in the multivariate Cox analysis adjusted for standard prognostic indicators, the PTEN-16p13.3 co-alteration status remained significant and conferred the highest risk of biochemical recurrence (hazard ratio: 4.18, 95% confidence interval: 1.82-9.59; P=0.001; Table 9, (A)). Similarly, a PTEN deletion along a 16p13.3 gain increased the risk of recurrence significantly even after adjusting for the CAPRA-S score risk groups (hazard ratio: 4.70, 95% confidence interval: 2.12-10.42, P<0.0001, Table 9, (B)). To estimate the potential prognostic benefit of assessing both genomic alterations, the C-index was calculated for each of PTEN deletion and 16p13.3 gain alone and in combination using biochemical recurrence as an endpoint in Cox model. The C-index was higher by using both alterations than 16p13.3 gain or PTEN deletion alone (0.69 vs. 0.62 and 0.63, respectively) and each of these alterations improved the C-index of the CAPRA-S score reaching a maximum when both were included (0.78, FIG. 8D).

TABLE 9
Univariate and multivariate Cox proportional hazard analysis predicting
biochemical recurrence for the PTEN-16p13 co-alteration status adjusted
for (A) standard clinicopathologic parameters and (B) CAPRA-S score.

| | Unicariate analysis | | Univariate analysis | |
|---|---|---|---|---|
| | Hazard ratio (95% | | Hazard ratio (95% | |
| Variables | confidence interval) | P-value | confidence interval) | P-value |

(A) Standard clinicopathologic parameters
PTEN-16p13 co-alteration status

| No PTEN and No 16p13 | — | | <0.0001 | — | | 0.03 |
|---|---|---|---|---|---|---|
| (reference) | | | | | | |
| PTEN or 16p13 | 3.93 | (1.88-8.25) | <0.0001 | 2.90 | (1.35-6.22) | 0.06 |
| PTEN and 16p13 | 6.88 | (3.14-15.08) | <0.0001 | 4.18 | (1.82-9.59) | 0.001 |
| Preoperative prostate- | 1.08 | (1.05-1.10) | <0.0001 | 1.05 | (1.02-1.08) | 0.002 |
| specific antigen^{a} | | | | | | |

Gleason grade

| | | | | | | |
|---|---|---|---|---|---|---|
| Group 3-5 (≥4 + 3) vs. | 4.70 | (2.79-7.90) | <0.0001 | 2.16 | (1.14-4.12) | 0.019 |
| group 1-2 (≤3 + 4) | | | | | | |
| pT-stage (T3 vs. T2) | 3.29 | (1.98-5.48) | <0.0001 | 1.46 | (0.80-2.67) | 0.217 |
| Surgical margin (positive vs | 2.25 | (1.37-3.72) | 0.002 | 1.50 | (0.87-2.57) | 0.143 |
| negative) | | | | | | |
| Age at surgery^{a} | 1.03 | (0.98-1.07) | 0.263 | 0.98 | (0.96-1.05) | 0.924 |

(B) CAPRA-S score
PTEN-16p13 co-alteration status

| No PTEN and no 16p13 | — | | <0.0001 | — | | 0.001 |
|---|---|---|---|---|---|---|
| (reference) | | | | | | |
| PTEN or 16p13 | 3.93 | (1.88-8.25) | <0.0001 | 3.55 | (1.69-7.46) | 0.001 |
| PTEN and 16p13 | 6.88 | (3.14-15.08) | <0.0001 | 4.70 | (2.12-10.42) | <0.0001 |

CAPRA-S risk

| Low (0-2) reference | — | | <0.0001 | — | | <0.0001 |
|---|---|---|---|---|---|---|
| Intermediate (3-5) | 3.21 | (1.66-6.19) | 0.001 | 2.70 | (1.40-5.23) | 0.003 |
| High (≥6) | 9.25 | (4.58-18.69) | <0.0001 | 7.34 | (3.57-15.07) | <0.0001 |

^{a}Analyzed as a continuous variable;
P-value: Wald test

In this example, the association of PTEN deletion with poor outcome in prostate cancer was confirmed and its potential at further stratifying low-intermediate risk patients treated by radical prostatectomy was demonstrated. In addition, it was shown that its prognostic value can be improved by considering the gain of 16p13.3. PTEN deletion was detected by FISH in 34% of the 287 radical prostatectomy specimens examined, a frequency falling within the range of 17-42% reported by other previously published studies using FISH and including over hundred samples (Krohn et al., 2012; Qu et al., 2016; Troyer et al., 2015; Yoshimoto et al., 2007; Bismar et al., 2011; Han et al., 2009). The majority of deletions observed were hemizygous (28% vs. 6% of homozygous) in agreement with most of the previous publications on radical prostatectomy cases. In contrast, Krohn et al. (2012) reported 12% of homozygous and 8% of hemizygous deletion in their cohort while Troyer et al. (2015) observed 9% homozygous and 9% hemizygous deletion in their samples. The variation in frequency of PTEN deletion and in proportion of hemizygous vs. homozygous deletion observed among the studies possibly reflects differences of cohort sizes and clinicopathologic features, but also likely differences in tissue preparation and FISH scoring method. The presence of homozygous-deleted and hemizygous deleted nuclei in most of tumor classified as PTEN homozygous-deleted in this study likely reflect intratumoral heterogeneity and possibly disease progression.

Supporting a role for PTEN alteration in prostate cancer progression, this study showed that its deletion was significantly associated with the aggressive clinicopathologic features of high Gleason grade group and advanced surgical stage pT3, a finding consistent with previous reports of PTEN FISH on large radical prostatectomy sets (Krohn et al., 2012; Troyer et al., 2015). In agreement with prior report on a separate sample set (Choucair, Ejdelman, et al., 2012) as well as with previous studies of other groups (Krohn et al., 2012; Qu et al., 2016; Troyer et al., 2015; Yoshimoto et al., 2007), it was also shown that PTEN deletion assessed by FISH was associated with biochemical recurrence after radical prostatectomy. Moreover, the prognostic value of the deletion was independent of standard clinicopathologic markers. In this study, homozygous deletion was not associated with a higher risk of biochemical recurrence than hemizygous deletion in agreement with the report of Krohn et al. (2012), but in contrast to Yoshimoto et al. (2007) and Troyer et al. (2015). Present data indicate that the loss of one copy was sufficient to increase significantly the risk of biochemical recurrence, which is consistent with PTEN haploinsufficiency demonstrated in prostate cancer animal models (Kwabi-Addo et al., 2001). It is also possible that the second allele has been inactivated by alternative mechanisms (Whang et al., 1998), which was not investigated in this study. Interestingly, the PTEN deletion status could further stratify patients of low-intermediate risk grade group 1-2 (≤3+4), pT2, and prostate-specific antigen<10, a finding not reported in previous studies. The revised Gleason scoring system applied to the present cohort offers more refinement over previous iterations by splitting Gleason score 7 into two groups of distinct prognoses: grade group 2 (3+4) and grade group 3 (4+3). While the grade group 2 has the best outcome, it was further stratified by PTEN FISH. Since the percentage of Gleason pattern 4 was not recorded in this cohort, it is unclear if and how it would correlate with the PTEN status. Similarly, PTEN FISH was able to sub-classify cases belonging to low and intermediate CAPRA-S risk groups, thus emphasizing the potential complementary role of this marker to clinicopathologic assessment for outcome prediction.

PTEN deletions detected by FISH are known to be enriched in metastatic as compared to primary prostate tumors (Han et al., 2009; Qu et al., 2013). One PTEN FISH study reported metastasis outcome on radical prostatectomy specimens, but did not find any significant association with PTEN deletion (Troyer et al., 2015). Here, it was shown that patients with a PTEN deletion in their radical prostatectomy sample were at a higher risk of experiencing distant metastases. Present results are in agreement with Lotan et al. (2011) who used a selected high-risk radical prostatectomy cohort (all patients experienced a biochemical recurrence) to demonstrate that the loss of PTEN protein expression was associated with a shorter time to distant metastasis. While further validation on different cohorts is needed, present findings highlight the potential of PTEN deletion as a marker of disease progression to advanced metastatic disease.

As discussed above in Examples II and III, 16p13.3 gain is associated with aggressive clinicopathologic features of prostate cancer as well as an increased risk of biochemical recurrence and distant metastases in the same radical prostatectomy specimens surveyed here for PTEN deletion. Moreover, the 16p13.3 gain status improved the stratification of patients with intermediate and high risk of disease progression based on their CAPRA-S score. The analysis of the combined data presented here exemplified the advantages of considering both PTEN and 16p13.3 CNAs for biochemical recurrence risk stratification. Cases that were negative for PTEN deletion were further stratified by the 16p13.3 gain status and vice versa, thus allowing the identification of patients that have a reduced risk of biochemical recurrence. A maximum risk of biochemical recurrence was reached for patients whose tumors harbored both PTEN deletion and 16p13.3 gain. The advantage of this combinatorial approach was further evidenced by an increase of the C-index, which reached its maximum when coupled with the CAPRA-S score risk groups. Interestingly, the PTEN deletion status identified patients of CAPRA-S low-risk group who have an increased risk of biochemical recurrence, while the 16p13.3 gain status alone did not further stratify the low-risk group. These results are in agreement with previous studies showing that combinations of genomic features such as gene expression changes and copy number alterations, including PTEN deletion status, can add prognostic information to the CAPRA-S score (Cooperberg et al., 2015; Lennartz et al., 2016). Owing to the relatively small number of secondary adverse events like metastases and prostate cancer-specific deaths in this cohort, additional future studies can be performed focussing on assessing the clinical significance of this PTEN-16p13.3 co-alteration status with respect to these adverse clinical end-points in large independent cohorts with long clinical follow-up.

Given the enhanced risk of biochemical recurrence associated with co-alteration of PTEN and 16p13.3, patients with such tumors may benefit from adjuvant treatments. In prostate cancer, the usefulness of adjuvant systemic therapy such as chemotherapy postsurgery remains to be established (Pignot et al., 2018). The lack of demonstrated effectiveness may be explained in part by differences of tumor biology among patients. Molecular biomarkers such as PTEN/16p13.3 FISH assists in patient selection and thus improve the success of future clinical trials. The assessment of these markers retrospectively in samples of patients who have received adjuvant therapy may provide additional data to support this hypothesis.

PTEN inactivation occurs predominantly via genomic deletion at 10q23 (Cancer Genome Atlas Research Network, 2015; Cairns et al, 1997), which results in increased levels of phosphatidylinositol [3-5]-trisphosphate (PIP3). PIP3 triggers the phosphorylation and activation of AKT by PDK1 leading to the stimulation of pathways related to cell growth and survival (Toren et al., 2014). PDPK1 encoding PDK1 at 16p13.3 was identified as a potential driver of the gain and found that PDK1 stimulates prostate cancer cell migration in vitro (Choucair et al., 2012). Increasing data from the literature indicate that PDK1 not only phosphorylates AKT, but is also directly involved in cell invasion and migration (Gagliardi et al., 2015). It is possible that an overexpression of PDK1 resulting from 16p13.3 gain would potentialize the PIK3/AKT pathway activation and/or provide additional advantages relevant to tumor progression such as an increased cell motility leading to tumor cell spreading beyond the prostate, which may explain the augmented risk of tumor recurrence associated with both PTEN and 16p13.3 copy number alterations. Assessment of the in situ expression and activation status of the proteins involved in the PIK3/AKT pathway as well as further work on animal models are warranted to elucidate the role of PDK1 in PCa and validate this hypothesis.

Genomic instability, as reflected by the percentage of tumor genome harboring copy number alterations, has been shown to be associated with adverse outcome (Hieronymus et al., 2014; Lalonde et al., 2014). It is thus possible that the higher risk of biochemical recurrence associated with PTEN-16p13.3 co-alteration reflects an increased genomic instability. PTEN has been shown to contribute to genomic instability leading to aggressive prostate cancer in animal models (Hubbard et al., 2016). In previous CAN analysis, 8q24 gain (MYC) and 16q23 deletion along PTEN deletion and 16p13 gain was identified as the four most common alterations enriched in lymph node metastases (Lapointe et al., 2007). It has been shown that the co-deletion of PTEN and 16q23 was associated with poor outcome after radical prostatectomy (Kluth et al., 2015). These other key copy number alterations may provide additional prognostic value, in particular MYC that synergizes with PTEN for tumor initiation and progression in animal models (Hubbard et al., 2016).

One use for such a panel of copy number alterations would be as FISH biomarkers on diagnostic biopsies to identify more effectively patients suitable for active surveillance, ultimately improving the pretreatment prognostication given that accurate Gleason grade on biopsies can be challenging to obtain. PTEN deletion detected in prostate needle biopsies of Gleason score 6 (grade group 1) has been shown to be associated with upgrading to Gleason score 7+(grade group 2 and up) at radical prostatectomy (Picanco-Albuquerque et al., 2016), which suggests that molecular biomarkers may overcome some of the limitations associated with the standard histopathologic evaluation of biopsy specimens. The improved patient risk stratification afforded by combining PTEN/16p13.3 may also be applicable in biopsy specimens from an active surveillance cohort.

FISH is regarded as the gold standard for the assessment of copy number alterations in tissue specimens owing to its ability to delineate specific genomic events with a spatial resolution facilitating a sensitive evaluation of individual cancer foci at a single-cell level (Bishop et al., 2010; Gozzetti et al., 2000). Assessing the expression of encoded proteins by immunohistochemistry is considered as an alternative approach to capture the prognostic value associated with copy number alterations. Previous PTEN immunohistochemistry assays applied to large cohorts have yielded variable results in terms of outcome prediction and correlation with FISH (Krohn et al., 2012; Cuzick et al., 2013). An improved PTEN immunohistochemistry assay was applied recently to one of the cohorts previously analyzed by immunohistochemistry and FISH mentioned above (Krohn et al., 2012) and the authors reported a sensitivity of 83% and 67% to respectively detect homozygous and hemizygous deletion (Lotan et al., 2017). While PTEN protein loss assessed by immunohistochemistry was associated with increased risk of biochemical recurrence, a further risk stratification was achieved by combining FISH with immunohistochemistry results, substantiating the enhanced value of PTEN FISH in outcome prediction. Future studies comparing both FISH and immunohistochemistry on additional cohorts, including present cohort, would be important to confirm these observations.

In summary, the results of this study support the prognostic value of PTEN deletion in prostate cancer, which can be further improved in combination with 16p13.3 gain status, suggesting that these genomic alterations may cooperatively contribute to prostate cancer progression. DNA copy number analysis of PTEN and 16p13.3 could be of important clinical value particularly for preoperative risk assessment of the clinically most challenging group of low-grade and intermediate-grade prostate cancer.

### REFERENCES

Abel F, Ejeskar K, Kogner P, Martinsson T. Gain of chromosome arm 17q is associated with unfavourable prognosis in neuroblastoma, but does not involve mutations in the somatostatin receptor 2(SSTR2) gene at 17q24. Br J Cancer 1999; 81:1402-9.
An G, Xu Y, Shi L, Shizhen Z, Deng S, Xie Z, et al. Chromosome 1q21 gains confer inferior outcomes in multiple myeloma treated with bortezomib but copy number variation and percentage of plasma cells involved have no additional prognostic value. Haematologica 2014; 99:353-9.
Beltran H, Prandi D, Mosquera J M, Benelli M, Puca L, Cyrta J, et al. Divergent clonal evolution of castration-resistant neuroendocrine prostate cancer. Nat Med 2016; 22:298-305.
Bishop R. Applications of fluorescence in situ hybridization (FISH) in detecting genetic aberrations of medical significance. Biosci Horiz 2010; 3: 85-95.
Bismar T A, Yoshimoto M, Vollmer R T, Duan Q, Firszt M, Corcos J, et al. PTEN genomic deletion is an early event associated with ERG gene rearrangements in prostate cancer. BJU international 2011; 107:477-85.
Bown N, Cotterill S, Lastowska M, O'Neill S, Pearson A D, Plantaz D, et al. Gain of chromosome arm 17q and adverse outcome in patients with neuroblastoma. N Engl J Med 1999; 340:1954-61.
Cancer Genome Atlas Research Network. The Molecular Taxonomy of Primary Prostate Cancer. Cell 2015; 163:1011-25.
Cairns P, Okami K, Halachmi S, Halachmi N, Esteller M, Herman J G, et al. Frequent inactivation of PTEN/MMAC1 in primary prostate cancer. Cancer Res 1997; 57:4997-5000.
Chang A J, Autio K A, Roach M, 3rd, Scher H I. High-risk prostate cancer-classification and therapy. Nat Rev Clin Oncol 2014; 11:308-23.
Chapuy B, Koch R, Radunski U, Corsham S, Cheong N, Inagaki N, et al. Intracellular ABC transporter A3 confers multidrug resistance in leukemia cells by lysosomal drug sequestration. Leukemia 2008; 22:1576-86.
Choucair K A, Guerard K P, Ejdelman J, Chevalier S, Yoshimoto M, Scarlata E, et al. The 16p13.3 (PDPK1) genomic gain in prostate cancer: a potential role in disease progression. Transl Oncol 2012; 5:453-60.
Choucair K, Ejdelman J, Brimo F, Aprikian A, Chevalier S, Lapointe J. PTEN genomic deletion predicts prostate cancer recurrence and is associated with low AR expression and transcriptional activity. BMC Cancer 2012; 12:543.
Cooperberg M R, Hilton J F, Carroll P R. The CAPRA-S score: a straightforward tool for improved prediction of outcomes after radical prostatectomy. Cancer 2011; 117:5039-46.
Cooperberg M R, Davicioni E, Crisan A, Jenkins R B, Ghadessi M, Karnes R J. Combined value of validated clinical and genomic risk stratification tools for predicting prostate cancer mortality in a high-risk prostatectomy cohort. Eur Urol 2015; 67:326-33.
Cuzick J, Berney D M, Fisher G, Mesher D, Moller H, Reid J E, et al. Prognostic value of a cell cycle progression signature for prostate cancer death in a conservatively managed needle biopsy cohort. Br J Cancer 2012; 106:1095-9.
Epstein J I, Feng Z, Trock B J, Pierorazio P M. Upgrading and downgrading of prostate cancer from biopsy to radical prostatectomy: incidence and predictive factors using the modified Gleason grading system and factoring in tertiary grades. European urology 2012; 61:1019-24.
Epstein J I, Egevad L, Amin M B, Delahunt B, Srigley J R, Humphrey P A, et al. The 2014 International Society of Urological Pathology (ISUP) Consensus Conference on Gleason Grading of Prostatic Carcinoma: Definition of Grading Patterns and Proposal for aNewGrading System. AmJ Surg Pathol 2016; 40:244-52.
Gagliardi P A, di Blasio L, Primo L. PDK1: A signaling hub for cell migration and tumor invasion. Biochimica et biophysica acta 2015; 1856:178-88.
Gozzetti A, Le Beau M M. Fluorescence in situ hybridization: uses and limitations. Seminars in Hematology 2000; 37:320-33.
Han B, Mehra R, Lonigro R J, Wang L, Suleman K, Menon A, et al. Fluorescence in situ hybridization study shows association of PTEN deletion with ERG rearrangement during prostate cancer progression. Mod Pathol 2009; 22:1083-93.
Harrell F E, Lee K L, Mark D B. Multivariable prognostic models: issues in developing models, evaluating assumptions and adequacy, and measuring and reducing errors. Stat Med 1996; 15:361-87.
Hieronymus H, Schultz N, Gopalan A, Carver B S, Chang M T, Xiao Y, et al. Copy number alteration burden predicts prostate cancer relapse. Proc Natl Acad Sci USA 2014; 111: 11139-44.
Hubbard G K, Mutton L N, Khalili M, McMullin R P, Hicks J L, Bianchi-Frias D, et al. Combined MYC Activation and Pten Loss Are Sufficient to Create Genomic Instability and Lethal Metastatic Prostate Cancer. Cancer Res 2016; 76:283-92.
Humphrey P A, Moch H, Cubilla A L, Ulbright T M, Reuter V E. The 2016 WHO Classification of Tumours of the Urinary System and Male Genital Organs-Part B: Prostate and Bladder Tumours. European urology 2016; 70:106-19.
Irshad S, Bansal M, Castillo-Martin M, Zheng T, Aytes A, Wenske S, et al. A molecular signature predictive of indolent prostate cancer. Sci Transl Med 2013; 5:202ra122.
Klein E A, Cooperberg M R, Magi-Galluzzi C, Simko J P, Falzarano S M, Maddala T, et al. A 17-gene assay to predict prostate cancer aggressiveness in the context of Gleason grade heterogeneity, tumor multifocality, and biopsy undersampling. Eur Urol 2014; 66:550-60.
Klein E A, Haddad Z, Yousefi K, Lam L L, Wang Q, Choeurng V, et al. Decipher genomic classifier measured on prostate biopsy predicts metastasis risk. Urology 2016; 90:148-52.
Klotz L, Vesprini D, Sethukavalan P, Jethava V, Zhang L, Jain S, et al. Long-term follow-up of a large active surveillance cohort of patients with prostate cancer. Journal of Clinical Oncology 2014; 33:272-7.
Kluth M, Runte F, Barow P, Omari J, Abdelaziz Z M, Paustian L, et al. Concurrent deletion of 16q23 and PTEN is an independent prognostic feature in prostate cancer. Int J Cancer 2015; 137:2354-63.
Krohn A, Diedler T, Burkhardt L, Mayer P S, De Silva C, Meyer-Kornblum M, et al. Genomic deletion of PTEN is associated with tumor progression and early PSA recurrence in ERG fusionpositive and fusion-negative prostate cancer. Am J Pathol 2012; 181:401-12.
Kwabi-Addo B, Gini D, Schmidt K, Podsypanina K, Parsons R, Greenberg N, et al. Haploinsufficiency of the Pten tumor suppressor gene promotes prostate cancer progression. Proc Natl Acad Sci USA 2001; 98:11563-8.
Lalonde E, Ishkanian A S, Sykes J, Fraser M, Ross-Adams H, Erho N, et al. Tumour genomic and microenvironmental heterogeneity for integrated prediction of 5-year biochemical recurrence of prostate cancer: a retrospective cohort study. The Lancet 2014; 15:1521-32.
Lapointe J, Li C, Giacomini C P, Salari K, Huang S, Wang P, et al. Genomic profiling reveals alternative genetic pathways of prostate tumorigenesis. Cancer Res 2007; 67:8504-10.
Lennartz M, Minner S, Brasch S, Wittmann H, Paterna L, Angermeier K, et al. The combination of DNA ploidy status and PTEN/6q15 deletions provides strong and independent prognostic information in prostate cancer. Clin Cancer Res 2016; 22:2802-11.
Lotan T L, Gurel B, Sutcliffe S, Esopi D, Liu W, Xu J, et al. PTEN protein loss by immunostaining: analytic validation and prognostic indicator for a high risk surgical cohort of prostate cancer patients. Clin Cancer Res 2011; 17:6563-73.
Lotan T L, Heumann A, Rico S D, Hicks J, Lecksell K, Koop C, et al. PTEN loss detection in prostate cancer: comparison of PTEN immunohistochemistry and PTEN FISH in a large retrospective prostatectomy cohort. Oncotarget 2017; 8:65566-76.
Mampaey E, Fieuw A, Van LaethemT, Ferdinande L, Claes K, Ceelen W, et al. Focus on 16p13.3 locus in colon cancer. PLoS One 2015; 10:e0131421.
Marshall J C, Nantel A, Blanco P, Ash J, Cruess S R, Burnier M N. Transcriptional profiling of human uveal melanoma from cell lines to intraocular tumors to metastasis. Clin Exp Metastasis 2007; 24:353-62.
Maurer M, Su T, Saal L H, Koujak S, Hopkins B D, Barkley C R, et al. 3-Phosphoinositide-dependent kinase 1 potentiates upstream lesions on the phosphatidylinositol 3-kinase pathway in breast carcinoma. Cancer Res 2009; 69:6299-306.
McShane L M, Altman D G, SauerbreiW, Taube S E, GionM, Clark G M, et al. REporting recommendations for tumour MARKer prognostic studies (REMARK). Br J Cancer 2005; 93:387-91.
Moyzis R K, Albright K L, Bartholdi M F, Cram L S, Deaven L L, Hildebrand C E, et al. Human chromosome-specific repetitive DNA sequences: novel markers for genetic analysis. Chromosoma 1987; 95:375-86.
Picanco-Albuquerque C G, Morais C L, Carvalho F L, Peskoe S B, Hicks J L, Ludkovski O, et al. In prostate cancer needle biopsies, detections of PTEN loss by fluorescence in situ hybridization (FISH) and by immunohistochemistry (IHC) are concordant and show consistent association with upgrading. Virchows Arch 2016; 468:607-17.
Pignot G, Maillet D, Gross E, Barthelemy P, Beauval J B, Constans-Schlurmann F, et al. Systemic treatments for high-risk localized prostate cancer. Nat Rev Urol 2018.
Punnen S, Freedland S J, Presti J C, Aronson W J, Terris M K, Kane C J, et al. Multi-institutional validation of the CAPRA-S score to predict disease recurrence and mortality after radical prostatectomy. Eur Urol 2014; 65: 1171-7.
Qu X, Randhawa G, Friedman C, Kurland B F, Glaskova L, Coleman I, et al. A three-marker FISH panel detects more genetic aberrations of AR, PTEN and TMPRSS2/ERG in castrationresistant or metastatic prostate cancers than in primary prostate tumors. PLoS One 2013; 8:e74671.
Qu X, Jeldres C, Glaskova L, Friedman C, Schroeder S, Nelson P S, et al. Identification of Combinatorial Genomic Abnormalities Associated with Prostate Cancer Early Recurrence. The Journal of molecular diagnostics: JMD 2016; 18:215-24.
Shen H, Zhu Y, Wu Y J, Qiu H R, Shu Y Q. Genomic alterations in lung adenocarcinomas detected by multicolor fluorescence in situ hybridization and comparative genomic hybridization. Cancer Genet Cytogenet 2008; 181:100-7.
Steinbach D, Gillet J P, Sauerbrey A, Gruhn B, Dawczynski K, Bertholet V, et al. ABCA3 as a possible cause of drug resistance in childhood acute myeloid leukemia. Clin Cancer Res 2006; 12:4357-63.
Tamoto E, Tada M, Murakawa K, Takada M, Shindo G, Teramoto K, et al. Gene-expression profile changes correlated with tumor progression and lymph node metastasis in esophageal cancer. Clin Cancer Res 2004; 10: 3629-38.
Taylor B S, Schultz N, Hieronymus H, Gopalan A, Xiao Y, Carver B S, et al. Integrative genomic profiling of human prostate cancer. Cancer Cell 2010; 18:11-22.
Toren P, Zoubeidi A. Targeting the PI3K/Akt pathway in prostate cancer: challenges and opportunities (review). Int J Oncol 2014; 45:1793-801.
Towle R, Tsui I F, Zhu Y, MacLellan S, Poh C F, Garnis C. Recurring DNA copy number gain at chromosome 9p13 plays a role in the activation of multiple candidate oncogenes in progressing oral premalignant lesions. Cancer Med 2014; 3:1170-84.
Troyer D A, Jamaspishvili T, Wei W, Feng Z, Good J, Hawley S, et al. A multicenter study shows PTEN deletion is strongly associated with seminal vesicle involvement and extracapsular extension in localized prostate cancer. Prostate 2015; 75:206-15.
Valk K, Vooder T, Kolde R, Reintam M A, Petzold C, Vilo J, et al. Gene expression profiles of non-small cell lung cancer: survival prediction and new biomarkers. Oncology 2010; 79:283-92.
Whang Y E, Wu X, Suzuki H, Reiter R E, Tran C, Vessella R L, et al. Inactivation of the tumor suppressor PTEN/MMAC1 in advanced human prostate cancer through loss of expression. Proc Natl Acad Sci USA 1998; 95:5246-50.
Williams C, Edvardsson K, Lewandowski S A, Strom A, Gustafsson J A. A genome-wide study of the repressive effects of estrogen receptor beta on estrogen receptor alpha signaling in breast cancer cells. Oncogene 2008; 27:1019-32.
Yasui K, Mihara S, Zhao C, Okamoto H, Saito-Ohara F, Tomida A, et al. Alteration in copy numbers of genes as a mechanism for acquired drug resistance. Cancer Res 2004; 64:1403-10.
Yoshimoto M, Cunha I W, Coudry R A, Fonseca F P, Torres C H, Soares F A, et al. FISH analysis of 107 prostate cancers shows that PTEN genomic deletion is associated with poor clinical outcome. British journal of cancer 2007; 97:678-85.

What is claimed is:

1. A method of prognosing a subject with prostate cancer, the method comprising:
(a) providing a first sample from the prostate from the subject suspected of comprising cancer cells;
(b) contacting the first sample with a first probe capable of specifically recognizing a 16p13.3 chromosome region, wherein the first probe is associated with a first label;
(c) contacting the first sample with a first reference probe capable of specifically recognizing a reference region of chromosome 16, wherein the first reference probe is associated with a first reference label;
(d) detecting the signal from the first label and from the first reference label; and
(e) classifying the cancer risk of the subject based on the presence or absence of a 16p13.3 gain, wherein the 16p13.3 gain is detected if the signal from the first label of the first probe is greater than the signal from the first reference label of the first reference probe.

2. The method of claim 1, further comprising:
(f) providing a second sample from the prostate from the subject suspected of comprising cancer cells;
(g) contacting the second sample with a second probe capable of specifically recognizing a 10q23.3 (PTEN) chromosome region, wherein the second probe is associated with a second label;
(h) contacting the second sample with a second reference probe capable of specifically recognizing a reference region of chromosome 10, wherein the second reference probe is associated with a second reference label;
(i) detecting the signal from the second label and from the second reference label; and
(j) classifying the cancer risk of the subject based on the presence or absence of a PTEN deletion, wherein the PTEN deletion is detected if the signal from the second label is less than the signal from the second reference label.

3. The method of claim 1, wherein the sample is a tumor sample.

4. The method of claim 2, wherein the first sample is the second sample.

5. The method of claim 1, wherein the subject presents low to intermediate risk clinical features.

6. The method of claim 1, wherein the first probe is derived from a RP11-20123 DNA clone, variants thereof, fragments thereof, or complements thereof.

7. The method of claim 1, wherein the first probe is capable of hybridizing to one or more genes of the 16p13.3 chromosome region comprising PKD1, RAB26, TRAF7, CASKIN1, MLST8, PGP, E4F1, DNASE1L2, ECI1, RNPS1, ABCA3, ABCA17A, CCNF, NTN3, TBC1D24, ATP6V0C, AMDHD2, CEMP1, PDPK1, variants thereof, fragments thereof, or complements thereof.

8. The method of claim 1, wherein the first reference probe is capable of specifically recognizing a 16qh centromeric region.

9. The method of claim 1, wherein the first reference probe is a derived from a pHuR-195 DNA clone, variants thereof, fragments thereof, or complements thereof.

10. The method of claim 2, wherein the second probe is derived from a CTD-2557P6 DNA clone, variants thereof, fragments thereof, or complements thereof.

11. The method of claim 2, wherein the second reference label is associated with a second reference probe capable of specifically recognizing a 10p11.1-q11.1 centromeric region.

12. The method of claim 2, wherein the second reference probe is a CEP10 probe, variants thereof, fragments thereof, or complements thereof.

13. The method of claim 1, wherein the label is a fluorescent label.

14. The method of claim 13, comprising detecting the fluorescent labels with fluorescent in situ hybridization (FISH) method.

15. The method of claim 1, further comprising treating the subject with an adjuvant or a neoadjuvant therapy if the subject is characterized as being associated with poor prognosis.

16. The method of claim 15, wherein the adjuvant or neoadjuvant therapy comprises surgery, radiation therapy, hormonotherapy and/or chemotherapy.

17. The method of claim 1, wherein the subject is a human.

18. The method of claim 1, wherein the subject has previously had a radical prostatectomy.

19. The method of claim 1, wherein the sample is from a resected tissue or from a biopsy.
